SEQUENCE LISTING (3 sequences)

SEQ ID NO: 1 - protein, 18 aa, Artificial
  Apolipoprotein A1 memetic
DWLKAFYDKVAEKLKEAF

SEQ ID NO: 2 - protein, 18 aa, Artificial
  Apolipoprotein A1 mimetic peptide
DWFKAFYDKVAEKFKEAF

SEQ ID NO: 3 - protein, 18 aa, Artificial
  Apolipoprotein A1 mimetic peptide
FAEKFKEAVKDYFAKFWD

(19) United States
(12) Patent Application Publication
(10) Pub. No.: US 2017/0128591 A1
(43) Pub. Date: May 11, 2017

(54) Peptide Containing Porphyrin Lipid Nanovesicles

(71) Applicant: UNIVERSITY HEALTH NETWORK, Toronto (CA)

(72) Inventors: Juan Chen, Toronto (CA); Gang Zheng, Toronto (CA); Liyang Cui, Palo Alto, CA (US)

(21) Appl. No.: 15/319,607

(22) PCT Filed: Jun. 18, 2015
(86) PCT No.: PCT/CA2015/000397

§ 371 Date: Dec. 16, 2016

Related U.S. Application Data
(60) Provisional application No. 62/014,964, filed on Jun. 20, 2014.

(51) Int. Cl. A61K 9/16; B82Y 5/00; A61K 49/00; A61K 31/404; A61K 9/51; A61K 41/00

(52) U.S. Cl. CPC A61K 47/48807; A61K 47/48053; A61K 47/48069; A61K 9/5169; B82Y 5/00; A61K 47/48215; A61K 41/0076; A61K 49/0036; A61K 31/404; Y10S 977/906; Y10S 977/915; Y10S 977/773; Y10S 977/801

(57) ABSTRACT

There is provided herein a nanovesicle comprising a monolayer of phospholipid, porphyrin-phospholipid conjugate and a peptide encapsulating a hydrophobic core, wherein the peptide comprises an amino acid sequence capable of forming at least one amphipathic α-helix; the porphyrin-phospholipid conjugate comprises one porphyrin, porphyrin derivative or porphyrin analog covalently attached to a lipid side chain, preferably at the sn-1 or the sn-2 position, of one phospholipid; the molar % of porphyrin-phospholipid conjugate to phospholipid is 35% or less; the nanovesicle is 35 nm in diameter or less.

### RELATED APPLICATIONS

This application claims priority to U.S. Provisional Application No. 62/014,964.

### FIELD OF THE INVENTION

The invention relates to nanovesicles, and more specifically to nanovesicles comprising phospholipid, porphyrin-phospholipid conjugate and a peptide encapsulating a hydrophobic core.

### BACKGROUND OF THE INVENTION

In recent years, multifunctional nanoparticles have been developed for many applications such as biosensors, diagnostic nanoprobes, and targeted drug delivery. The efforts have been driven to a large extent by the need to improve biological specificity in diagnosis and therapy. Porphyrins, which are pigments from chlorophyll, and their derivatives have proved particular success for photodynamic therapy (PDT) and fluorescence imaging of cancer.(1-4) However, their poor solubility in aqueous solution at physiological condition prevents their clinic application.(5) Continuous efforts have been devoted to encapsulate or attach these hydrophobic photosensitizers to various nanoparticles, including liposomes, polymeric, gold and silica nanoparticles to improve their systemic delivery efficiency.(6-8) However, the encapsulation method has limitation on carrying the porphyrin molecules, for example the liposome only can carry less than 15 molar % to keep the nanostructure stable.(6)

Recently, we have developed a porphysome nanostructure self-assembled by even 100% porphyrin-phospholipid conjugates.(9) The stable nanostructure (100-150 nm diameter) with high density of porphyrin molecules fully arranged in the liposome-like bilayer membrane offers novel biophotonic functions to porphysome beyond porphyrins monomers. Its nanostructure-dependent ‘super’-absorption (extinction coefficient ε₆₈₀=2.9×10⁹M⁻¹cm⁻¹) and ‘super’-quenching of photoactivity convert light energy to heat with extremely high efficiency, giving them ideal photothermal and photoacoustic properties that are unprecedented for organic nanoparticles. The receptor-mediated nanoparticle uptake facilitates the porphysome intracellular internalization and nanostructure disruption, resulting in the restoration of photoactivity of porphyrin for non-invasive fluorescence imaging and effective PDT.(10) In addition, radioactive copper-64 (⁶⁴Cu) can be directly incorporated into the porphyrin-lipid building blocks of the preformed porphysomes for non-invasive PET imaging.(11-12) Thus, the intrinsic multimodal nature of porphyrin-assembled nanoparticles confers high potential for cancer theranostics and clinical translation.

Porphysome in the 100-150 nm size range exhibits preferential accumulation in malignant tumors through the enhanced permeability and retention (EPR) effect, but may encounter the diffusive hindrance for sufficient penetration within tumor. Recent studies have demonstrated that nanoparticles less than 40 nm displayed more effective at penetrating deeply into fibrous tumors than their larger counterparts.(13-15) For example, Cabral et al compared the accumulation and effectiveness of different sizes of drug-loaded polymeric micelles (with diameters of 30, 50, 70 and 100 nm) in both highly and poorly permeable tumors. All the polymer micelles penetrated highly permeable tumors in mice, but only the 30 nm micelles could penetrate poorly permeable pancreatic tumors to achieve an antitumour effect. (14) Thus, the development of porphyrin nanoparticles with smaller size (<30 nm) has potential to enhance their diffusive transport through the tumor interstitium, especially in the tumor with low permeability, allowing efficient penetration and accumulation to reach therapeutically relevant concentrations. However, attempts to create smaller porphysome by the self-assembly of phophyrin-lipid remain a challenge due to growing instability as a result of the surface curvature.

Further, applicant refers to previous PCT Patent Publication Nos. 11/044671, 12/167350, 13/053042, 13/082702, 13/159185, 14/000062, and 09/073984, all of which are hereby incorporated by reference.

### SUMMARY OF THE INVENTION

In an aspect, there is provided a nanovesicle comprising a monolayer of phospholipid, porphyrin-phospholipid conjugate and a peptide encapsulating a hydrophobic core, wherein
the peptide comprises an amino acid sequence capable of forming at least one amphipathic α-helix;
the porphyrin-phospholipid conjugate comprises one porphyrin, porphyrin derivative or porphyrin analog covalently attached to a lipid side chain, preferably at the sn-1 or the sn-2 position, of one phospholipid;
the molar % of porphyrin-phospholipid conjugate to phospholipid is 35% or less;
the nanovesicle is 35 nm in diameter or less.

In an aspect, there is provided a method of imaging on a target area in a subject comprising: providing the nanovesicle described herein; administering the nanovesicle to the subject; and imaging the target area.

In an aspect, there is provided use of the nanovesicle described herein for performing imaging on a target area in a subject, preferably a tumour.

In an aspect, there is provided a method of performing photodynamic on a target area in a subject comprising: providing the nanovesicle described herein; administering the nanovesicle to the subject; and irradiating the nanovesicle at the target area with a wavelength of light, wherein the wavelength of light activates the porphyrin-phospholipid conjugate to generate singlet oxygen.

In an aspect, there is provided a method of delivering a hydrophobic agent to a subject comprising: providing the nanovesicle described herein, wherein the hydrophobic core comprises the agent; and administering the nanovesicle to the subject.

In an aspect, there is provided use of the nanovesicle described herein for delivering a hydrophobic agent performing imaging on a target area in a subject, wherein the hydrophobic core comprises the agent.

### BRIEF DESCRIPTION OF FIGURES

These and other features of the preferred embodiments of the invention will become more apparent in the following detailed description in which reference is made to the appended drawings wherein:

FIG. 1 (a) Sizes in diameter (volume distribution peak) of formulations with various pyro initial input (pyrolipid/total phospholipid=5%, 10%, 30% and 50%) after 0.1 μm filtration. (b) Porphyrin fluorescence quenching efficiency for each formulation. % Quenching=(1-FI_{intact in PBS}/FI_{disrupted by Triton})×100%.

FIG. 2 shows size distribution by volume and TEM images of USPVs.

FIG. 3 UV-vis and CD spectra of USPV.

FIG. 4 shows fluorescence spectra and singlet oxygen generation of (a) porphysome and (b) USPV, intact in PBS or disrupted by Triton X-100.

FIG. 5 shows (a) Cell uptake of porphysomes vs. USPVs in U87 cells measured by cell lysis assay. (b) Confocal imaging of cells incubated with porphysome and USPV (10 μM pyrolipid, 3 h incubation).

FIG. 6 shows blood clearance profile of USPV, PEG-USPV and folate-PEG-USPV.

FIG. 7 shows bioluminescence images (left panel) and in situ fluorescence images (centered panel) and white light photos (right panel) of 9L^{luc} glioma-bearing mice injected with (a) porphysomes and (b) USPVs at same pyrolipid concentration (200 nmol).

FIG. 8 shows (a) White image (left) and ex vivo fluorescence image (right) of the brain from 9L^{luc} glioma-bearing mouse, (b) corresponding H&E result confirming the regions of tumor (white dotted line squired area). (c) Microscopic image (left panel, blue: DAPI, red: pyro) of the frozen tissue slice from 9L^{luc} mice and corresponding H&E result (right panel) showing the same regions of tumor and contralateral healthy brain.

FIG. 9 shows (a) Size distribution by volume of USPV-DiR-BOA. (b) UV-Vis absorbance of USPV-DiR-BOA, (c) fluorescence spectra and (d) singlet oxygen generation of USPV-DiR-BOA, intact in PBS or disrupted by Triton X-100.

FIG. 10 shows (a) White light photos and corresponding in situ fluorescence images of U87 glioma-bearing mice injected with USPV-DiR-BOA at 24 h post intravenous injection. Both pyro channel (Ex: 575-605 nm, Em: 680-750 nm) and DiR-BOA channel (Ex: 725-755 nm, Em: 780-950 nm) were acquired. (b) Representative in vivo fluorescence microscopic images obtained with deep red long-pass (Ex: 660 nm, Em 689-900 nm) laser probe. With crania removed, both tumor and contralateral brain were examined. (c) Ex vivo fluorescence imaging of the major organs. Organs in the images are listed as follows, A: Muscle, B: Brain with tumor, C: Lung, D: Heart, E: Spleen, F: Kidneys, G: Liver.

FIG. 11 shows a) ⁶⁴Cu-USPV enable PET imaging of ovarian cancer metastases; ex vivo bioluminescence image b) and fluorescence image c) of metastases tumor and lymph nodes; the metastases tissue was confirmed by pancytokeratin (AE1/AE3) staining image d) and H&E staining image e).

FIG. 12 shows (a) Maestro imaging and fluorescence molecular tomography (FMT) imaging results of the brains with deep tumor expressing GFP. Imaging was performed 24 h post-injection. (b) Illustration of the brain transection. (c) Fluorescence imaging results with GFP channel, pyro channel and DiR-BOA channel.

FIG. 13 shows histology and tumor slice microscopic imaging results.

FIG. 14 shows white image, bioluminescence image and fluorescence image of brain with multi-foci after image-guided tumor removal.

FIG. 15 shows temperature monitoring during USPV-PDT treatment.

FIG. 16 shows H&E and TUNEL results of tumor area and surrounding brain in the laser control group and USPV-PDT treatment group with different light dose.

FIG. 17 shows TUNEL quantitative results of tumor and surrounding brain in USPV-PDT treatment group with different light dose.

FIG. 18 shows USPV-enabled non-invasive detection of primary tumor and lymphatic drainage in rabbit HNC model; a) Pharmacokinetic profile of USPV in HNC rabbits (n=4); b) Representative PET/CT 3D image of HNC rabbit at 24 h after intravenous injection of ⁶⁴Cu-USPV (red arrow: tumor, white arrow: regional lymph node); c) Distribution of ⁶⁴Cu-USPV in muscle, tumor and lymph node quantified by PET volumetric analysis. The uptake was presented as standard uptake values (SUV). Tumor and lymph node uptake of USPV were significantly higher than the muscle uptake (n=4, P<0.05); d) Distribution of ⁶⁴Cu-USPV in major organs in HNC rabbits (n=5) and healthy rabbits (n=3) measured by γ-counting; e) Ex vivo fluorescence of resected tumor, regional lymph node and other major organs of HNC rabbits after PET/CT imaging. LN represents lymph node and SG represents salivary gland.

FIG. 19 shows representative axial, sagittal and coronal views of 2D PET/CT imaging showing tumor (red arrow) and regional lymph node (white arrow).

FIG. 20 shows representative H&E, pancytokeratin staining and fluorescence microscopic imaging of the tumor (a) and metastatic lymph node (b) after 24 h intravenous injection of ⁶⁴Cu-USPV. (Scale bar: 100 mm).

FIG. 21 shows USPV-enabled fluorescence-guided resection of tumor and metastatic lymph nodes. In vivo fluorescence imaging of HNC tumor in rabbits at 24 h after intravenous injection of USPV: a) before incision with the skin intact; b) during surgery upon skin flap removal; c) post-surgery with the surgical bed non-fluorescent confirming the completion of the procedure; d) Representative H&E, Pancytokeratin staining and fluorescence microscopic imaging of the tissue slices of the resected tumor; e) Intra-operative fluorescence imaging of sentinel lymph node upon skin flap removal; f) Lymphatic network mapped by USPV fluorescence. A series of zoom-in images (position 1-5) were acquired followed the lymphatic flow from sentinel lymph node to regional lymph node; g) Representative H&E, pancytokeratin staining and fluorescence microscopic imaging of the tissue slices of the resected suspicious lymph nodes detected by USPV.

FIG. 22 shows USPV-enabled PDT in HNC rabbits. a) Illustration of the 2-step PDT laser irradiation at 24 h after intravenous injection of USPV; Representative photography (b) and axial CT images (c) of rabbits before and after USPV-PDT; d) Average tumor growth curve determined by volumetric CT measurement; Representative H&E and Pancytokeratin staining of the tissue resected from the original tumor region (e) and lymph node resected (f) at Day 34 after USPV-PDT. All tissues showed malignancy-free.

FIG. 23 shows the temperature change of tumors during laser irradiation. Temperature was monitored by thermal camera during laser irradiation of laser control group and USPV-PDT group.

FIG. 24 shows monitoring tumor size change by CT imaging after laser treatment. Representative CT sagittal image of laser control and USPV control group rabbits with tumor depicted after laser or USPV administration.

FIG. 25 shows representative CT sagittal images showing the regional lymph node of rabbits of USPV control, laser control and USPV-PDT group post-treatment.

FIG. 26 shows evaluation of the toxicity of USPV-PDT. a) Blood assay of rabbits before USPV administration and 1 week and 3 week after USPV-PDT treatment (n=4); b) Representative H&E staining sections of the main organs including heart, lung, liver, spleen, adrenal and muscle from USPV-PDT rabbits, indicating no side effect on healthy tissues after tumor ablation.

### DETAILED DESCRIPTION

In the following description, numerous specific details are set forth to provide a thorough understanding of the invention. However, it is understood that the invention may be practiced without these specific details.

Here, we introduced a novel ultra-small porphyrin vesicle (USPV) containing a hydrophobic drug core, enveloped by porphyrin lipid embedded phospholipid monolayer, and constrained by an ApoA-1 mimetic peptide network. We demonstrated that the α-helix structure formed by peptide network played essential role in constricting size and stabilizing the particles. Functionally like porphysome, USPV with 35% of porphyrin-lipid packing density has intrinsic multimodal biophotonic properties. The ultra small size nanostructure (<30 nm) drove sufficient absorption enhancement (extinction coefficient ε₆₈₀=7.8×10⁷ M⁻¹cm⁻¹) and efficient photoproperties quenching which resulted in the silence of porphyrin fluorescence and singlet oxygen generation. Therefore, the intact USPV is photodynamic inactive, while it will become PDT active when the nanostructure is disrupted. Meanwhile, the hydrophobic core of USPV can be loaded efficiently with hydrophobic bioactive drugs and its favorable blood circulation characteristics (10 h circulation half-life in mouse and 27 h in rabbit) present it as amiable drug delivery system without the need of PEGylation. Using a clinic relative mouse orthotopic glioma tumor model and a rabbit orthotopic head-and-neck cancer (HNC) rabbit model, we have demonstrated that USPV facilitated a stable and tumor-specific delivery of drug cargo. The ⁶⁴Cu labelled USPV enabled tracking of the in vivo fate of the nanoparticle and its drug cargos. The primary tumor, metastatic tumor and lymph nodes, and lymphatic drainage from tumor to regional lymph nodes could be visualized clearly by both pre-operative PET and intra-operative fluorescence imaging. Moreover, the effective photoproperties activation of the high-densely-packed porphyrin at 24 h post systemic administration allowed for a precise fluorescence-guided tumor resection and an effective PDT in both glioma mouse and HNC rabbit. It should be noted that this work is distinctively different from our previously reported porphysome in its nanostructure (20 nm vs. 100 nm, monolayer vs. bilayer, hydrophobic core vs. aqueous core, α-helical peptide vs. PEG coating) and nanostructure-dependent functions (fast vs. slow intracellular trafficking, photodynamic therapy vs. photothermal therapy).

In an aspect, there is provided a nanovesicle comprising a monolayer of phospholipid, porphyrin-phospholipid conjugate and a peptide encapsulating a hydrophobic core, wherein the peptide comprises an amino acid sequence capable of forming at least one amphipathic α-helix; the porphyrin-phospholipid conjugate comprises one porphyrin, porphyrin derivative or porphyrin analog covalently attached to a lipid side chain, preferably at the sn-1 or the sn-2 position, of one phospholipid; the molar % of porphyrin-phospholipid conjugate to phospholipid is 35% or less; the nanovesicle is 35 nm in diameter or less.

Suitable scaffold peptides may be selected from the group consisting of Class A, H, L and M α-helices or a fragment thereof. Suitable scaffold peptides may also comprise a reversed peptide sequence of the Class A, H, L and M amphipathic α-helices or a fragment thereof, as the property of forming an amphipathic α-helix is determined by the relative position of the amino acid residues within the peptide sequence.

In one embodiment, the scaffold peptide has an amino acid sequence comprising consecutive amino acids of an apolipoprotein, preferably selected from the group consisting of apoB-100, apoB-48, apoC, apoE and apoA.

The “amino acids” used in this invention, and the term as used in the specification and claims, include the known naturally occurring protein amino acids, which are referred to by both their common three letter abbreviation and single letter abbreviation. See generally Synthetic Peptides: A User's Guide, G A Grant, editor, W.H. Freeman & Co., New York, 1992, the teachings of which are incorporated herein by reference, including the text and table set forth at pages 11 through 24. As set forth above, the term “amino acid” also includes stereoisomers and modifications of naturally occurring protein amino acids, non-protein amino acids, post-translationally modified amino acids, enzymatically synthesized amino acids, derivatized amino acids, constructs or structures designed to mimic amino acids, and the like. Modified and unusual amino acids are described generally in Synthetic Peptides: A User's Guide, cited above; Hruby V J, Al-obeidi F and Kazmierski W: Biochem J 268:249-262, 1990; and Toniolo C: Int J Peptide Protein Res 35:287-300, 1990; the teachings of all of which are incorporated herein by reference.

“Alpha-helix” is used herein to refer to the common motif in the secondary structure of proteins. The alpha helix (α-helix) is a coiled conformation, resembling a spring, in which every backbone N—H group donates a hydrogen bond to the backbone C═O group of the amino acid four residues earlier. Typically, alpha helices made from naturally occurring amino acids will be right handed but left handed conformations are also known.

“Amphipathic” is a term describing a chemical compound possessing both hydrophilic and hydrophobic properties. An amphipathic alpha helix is an often-encountered secondary structural motif in biologically active peptides and proteins and refers to an alpha helix with opposing polar and nonpolar faces oriented along the long axis of the helix.

Examples of small amphipathic helix peptides include those described in WO 09/073984.

Methods for detecting and characterizing protein domains with putative amphipathic helical structure are set forth in Segrest, J. P. et al. in PROTEINS: Structure, Function, and Genetics (1990) 8:103-117, the contents of which are incorporated herein by reference. Segrest et al. have identified seven different classes of amphipathic helices and have identified peptides/proteins associated with each class. Of the seven different classes there are four lipid-associating amphipathic helix classes (A, H, L, and M). Of these, Class A, the designated apolipoprotein class, possesses optimal properties for forming phospholipid-based particles.

As used herein, “phospholipid” is a lipid having a hydrophilic head group having a phosphate group and hydrophobic lipid tail.

In some embodiments, the molar % of porphyrin-phospholipid conjugate to phospholipid is 35% or less, 30% or less, 25% or less, or 20-30%.

In some embodiments, the nanovesicle is substantially spherical and 35 nm in diameter or less, 25 nm in diameter or less, between 20-30 nm in diameter or about 25 nm in diameter.

In some embodiments, the porphyrin, porphyrin derivative or porphyrin analog in the porphyrin-phospholipid conjugate is selected from the group consisting of hematoporphyrin, protoporphyrin, tetraphenylporphyrin, a pyropheophorbide, a bacteriochlorophyll, chlorophyll a, a benzoporphyrin derivative, a tetrahydroxyphenyl chlorin, a purpurin, a benzochlorin, a naphthochlorins, a verdin, a rhodin, a keto chlorin, an azachlorin, a bacteriochlorin, a tolyporphyrin, a benzobacteriochlorin, an expanded porphyrin and a porphyrin isomer. Preferably, the expanded porphyrin is a texaphyrin, a sapphyrin or a hexaphyrin and the porphyrin isomer is a porphycene, an inverted porphyrin, a phthalocyanine, or a naphthalocyanine.

In some embodiments, the porphyrin in the porphyrin-phospholipid conjugate is pyropheophorbide-a acid.

In some embodiments, the porphyrin in the porphyrin-phospholipid conjugate is a bacteriochlorophyll derivate.

In some embodiments, the phospholipid in the porphyrin-phospholipid conjugate comprises phosphatidylcholine, phosphatidylethanoloamine, phosphatidylserine or phosphatidylinositol. Preferably, the phospholipid comprises an acyl side chain of 12 to 22 carbons.

In some embodiments, the phospholipid in the porphyrin-phospholipid conjugate is 1-Palmitoyl-2-Hydroxy-sn-Glycero-3-Phosphocholine or 1-Stearoyl-2-Hydroxy-sn-Glycero-3-Phosphocholine.

In some embodiments, the porphyrin-phospholipid conjugate is pyro-lipid.

In some embodiments, the porphyrin-phospholipid conjugate is oxy-bacteriochlorophyll-lipid.

In some embodiments, the porphyrin is conjugated to the glycerol group on the phospholipid by a carbon chain linker of 0 to 20 carbons.

In some embodiments, the porphyrin-phospholipid conjugate comprises a metal chelated therein, optionally a radioisotope of a metal, preferably selected from the group consisting of Zn, Cu, Mn, Fe and Pd.

In some embodiments, the phospholipid is an anionic phospholipid. Preferably, the phospholipid is selected from the group consisting of phosphatidylcholines, phosphatidylethanolamines, phosphatidic acid, phosphatidylglycerols and combinations thereof. In some embodiments, the phospholipid is selected from the group consisting of 1,2-dipalmitoyl-sn-glycero-3-phosphatidic acid (DPPA), 1,2-dipalmitoyl-sn-glycero-3-phosphatidylcholine (DPPC), 1,2-distearoyl-sn-glycero-3-phosphocholine (DSPC), 1,2-dimyristoyl-sn-glycero-3-phosphocholine (DMPC), 1,2-dibehenoyl-sn-glycero-3-phosphocholine (DBPC), 1,2-diarachidoyl-sn-glycero-3-phosphatidylcholine (DAPC), 1,2-dilignoceroyl-sn-glycero-3-phosphatidylcholine (DLgPC), 1,2-dipalmitoyl-sn-glycero-3-[phosphor-rac-(1-glycerol)] (DPPG) and combinations thereof.

In some embodiments, the peptide is selected from the group consisting of Class A, H, L and M amphipathic α-helices, fragments thereof, and peptides comprising a reversed peptide sequence of said Class A, H, L and M amphipathic α-helices or fragments thereof.

Preferably, the peptide consists of consecutive amino acids of an apoprotein, preferably selected from the group consisting of apoB-100, apoB-48, apoC, apoE and apoA.

In some embodiments, the peptide is selected from the group consisting of 2F (DWLKAFYDKVAEKLKEAF), 4F (DWFKAFYDKVAEKFKEAF), and the reverse sequences of the foregoing. In an embodiment, the peptide is the R4F peptide (Ac-FAEKFKEAVKDYFAKFWD).

In some embodiments, the at least one amphipathic α-helix or peptide is between 6 and 30 amino acids in length, 8 and 28 amino acids in length, 10 and 24 amino acids in length, 11 and 22 amino acids in length, 14 and 21 amino acids in length. 16 and 20 amino acids in length or 18 amino acids in length.

A wide variety of hydrophobic bioactive or therapeutic agents, pharmaceutical substances, or drugs can be encapsulated within the core of the USPV.

In some embodiments, the hydrophobic core comprises a hydrophobic diagnostic or therapeutic agent, preferably, paclitaxel, docetaxel, or 1,1′-dioctadecyl-3,3,3′,3′-tetramethylindotricarbocyanine iodide bis-oleate (DiR-BOA).

The term “therapeutic agent” is art-recognized and refers to any chemical moiety that is a biologically, physiologically, or pharmacologically active substance. Examples of therapeutic agents, also referred to as “drugs”, are described in well-known literature references such as the Merck Index, the Physicians' Desk Reference, and The Pharmacological Basis of Therapeutics, and they include, without limitation, medicaments; vitamins; mineral supplements; substances used for the treatment, prevention, diagnosis, cure or mitigation of a disease or illness; substances which affect the structure or function of the body; or pro-drugs, which become biologically active or more active after they have been placed in a physiological environment. Various forms of a therapeutic agent may be used which are capable of being released from the subject composition into adjacent tissues or fluids upon administration to a subject.

A “diagnostic” or “diagnostic agent” is any chemical moiety that may be used for diagnosis. For example, diagnostic agents include imaging agents, such as those containing radioisotopes such as indium or technetium; contrasting agents containing iodine or gadolinium; enzymes such as horse radish peroxidase, GFP, alkaline phosphatase, or β-galactosidase; fluorescent substances such as europium derivatives; luminescent substances such as N-methylacrydium derivatives or the like.

In some embodiments, the nanovesicle is PEG free.

In some embodiments, the nanovesicle further comprises PEG, preferably PEG-lipid, further preferably PEG-DSPE.

In some embodiments, the nanovesicle further comprises a targeting molecule.

In some embodiments, the nanovesicle further comprises targeting molecule, preferably an antibody, peptide, aptamer or folic acid.

“Targeting molecule” is any molecule that can direct the nanovesicle to a particular target, for example, by binding to a receptor or other molecule on the surface of a targeted cell. Targeting molecules may be proteins, peptides, nucleic acid molecules, saccharides or polysaccharides, receptor ligands or other small molecules. The degree of specificity can be modulated through the selection of the targeting molecule. For example, antibodies typically exhibit high specificity. These can be polyclonal, monoclonal, fragments, recombinant, or single chain, many of which are commercially available or readily obtained using standard techniques.

In an aspect, there is provided a method of imaging on a target area in a subject comprising: providing the nanovesicle described herein; administering the nanovesicle to the subject; and imaging the target area.

In an aspect, there is provided use of the nanovesicle described herein for performing imaging on a target area in a subject, preferably a tumour.

In an aspect, there is provided a method of performing photodynamic on a target area in a subject comprising a. providing the nanovesicle described herein; administering the nanovesicle to the subject; and irradiating the nanovesicle at the target area with a wavelength of light, wherein the wavelength of light activates the porphyrin-phospholipid conjugate to generate singlet oxygen.

In some embodiments, the target area is a tumour.

In an aspect, there is provided a method of delivering a hydrophobic agent to a subject comprising: providing the nanovesicle described herein, wherein the hydrophobic core comprises the agent; and administering the nanovesicle to the subject.

In an aspect, there is provided use of the nanovesicle described herein for delivering a hydrophobic agent performing imaging on a target area in a subject, wherein the hydrophobic core comprises the agent.

Possible advantages of the USPV when compared with traditional porphysomes include being smaller, less or no need for PEGylation for in vivo stability, enhanced singlet oxygen and fluorescence activation, and/or the ability to incorporate hydrophobic payload inside the core (e.g., drugs, CT contrast, etc.) and siRNA on the surface, while having porphysome functions (photo thermal, photo acoustic, PET, MRI, CT, etc.).

The advantages of the present invention are further illustrated by the following examples. The examples and their particular details set forth herein are presented for illustration only and should not be construed as a limitation on the claims of the present invention.

### EXAMPLES

Methods and Materials

Materials

1,2-dimyristoyl-sn-glycero-3-phosphocholine (DMPC), distearoyl-sn-glycero-3-phosphoethanolamine-N-methoxy(polyetheneglycol) (DSPE-PEG2000), and 1,2-distearoyl-sn-glycero-3-phosphoethanolamine-N-folate(polyethylene glycol) (folate-DSPE-PEG₂₀₀₀) were purchased from Avanti Polar Lipids Inc. (AL, USA). Cholesteryl oleate (CO) was obtained from Sigma-Aldrich Co. (MO, USA). 1,1′-dioctadecyl-3,3,3′,3′-tetramethylindotricarbocyanine iodide bis-oleate (DiR-BOA) and porphyrin-lipid (pyropheophorbide-lipid abbreviated as pyro-lipid) were prepared by previously reported protocols.(16) The ApoA-1 mimetic R4F peptide (R4F), Ac-FAEKFKEAVKDYFAKFWD, was purchased from GL Biochem Ltd. (Shanghai, China). Cell culture media Eagle's Minimum Essential Medium (EMEM) was obtained from the ATCC (American Type Culture Collection, Manassas, Va.). The fetal bovine serum (FBS), trypsin-ethylenediaminetetraacetic acid (EDTA) solution and Hoechst 33258 were all purchased from Gibco-Invitrogen Co. (CA, USA).

Ultra-Small Porphyrin Vesicles (USPV) Preparation and Characterization

Synthesis of USPV

A lipid film was prepared by evaporation of lipid mixtures in chloroform under nitrogen. The lipid mixture for USPV consists of 0.9 μmol porphyrin-lipid, 2.1 μmol DMPC and 0.3 μmol cholesterol oleate. For cargo-loaded particles, a 3 mol % DiR-BOA that serves as the model drug was added to the lipid mixture, for PEGylated USPV formulation (PEG-USPV), 1% DSPE-PEG₂₀₀₀ was added in the lipid mixture, and for folate receptor-targeted USPV (Folate-PEG-USPV), 1% folate-DSPE-PEG₂₀₀₀ was added in the lipid mixture. The completely dried lipid films were hydrated with 1.0 mL PBS buffer (150 mM, pH 7.5) and sonicated (Bioruptor®) at low frequency (30 s on/30 s off) for 30 cycles at 40° C. R4F peptide (2.3 mg, 5 mg/ml) was titrated into the rehydrated solution and the turbid emulsion became transparent upon the addition of the peptide solution. The mixture was kept shaking at 4° C. overnight. The solution was centrifuged at 12,000 rpm for 20 min subsequently and the supernatant was filtered with 0.1 μm membrane (Millex®, Sigma-Aldrich).

Size and Morphology of USPV

The size distribution and (potential of USPV were measured by dynamic light scattering (ZS90 Nanosizer, Malvern Instruments). Transmission electron microscopy (TEM) with Hitachi (Japan) H-7000 electron microscope was used to determine the particle morphology and the size.

Excitation and Emission of USPV

USPVs were diluted with either PBS as intact/quenched samples or 0.5% Triton X-100 in PBS as disrupted/unquenched samples. The absorption spectra of the intact and disrupted USPV were measured by UV/Vis spectrophotometer Cary 50 (Agilent, Mississauga, ON) and their fluorescence were measured by using Fluoromax-4 fluorometer (Horiba Jobin Yvon, USA) (Excitation: 420 nm, Emission: 600-800 nm, slit width: 5 nm). The fluorescence quenching efficiency was calculated by the following formula: (1-FI_{intact}/FI_{disrupted})×100%, (FI_{intact} and FI_{disrupted} represent the fluorescence intensity of intact sample and disrupted sample respectively.

Singlet Oxygen (¹O₂) Generation of USPV

¹O₂ generation of USPVs (both intact and disrupted) were measured using SOSG assay. Briefly, a SOSG (¹O₂ sensor green reagent, Molecular Proves, Inc.) solution was freshly prepared in methanol (5 mM) and mixed with USPV (final pyro concentration at 1 μM), intact in PBS or disrupted in 0.5% Triton X-100, to have a final SOSG concentration of 6 μM. Samples were treated with an array of light-emitting diodes at 671 nm with light fluence from 0.5 J/cm² to 10 J/cm², and SOSG fluorescence was then measured by exciting at 504 nm and collecting at 525 nm. There was no porphyrin fluorescence contribution within this emission window.

Quantitative Cellular Uptake Study and Confocal Microscopy

U87^{GFP} and U87^{luc} cells were cultured in Eagle's Minimum Essential Medium (EMEM, ATCC®) with 10% FBS. To compare the cellular uptake of USPV versus porphysome, a quantitative cellular uptake study was performed on U87 glioma cells. Briefly, U87^{GFP} cells were seeded in 6-well plate at 10⁶ cells per well 24 h prior to incubation and incubated with porphysome and USPV at the porphyrin concentration of 10 μM for 3 h at 37° C. Following 3 times rinse with PBS, the cells were trypsinized and the suspension was centrifuged at 4000 rpm for 5 min. The cell pellets were then re-suspended in 500 μL lysis buffer and incubated on ice for 1 h. The solution was centrifuged at 10,000 rpm for 10 min and the supernatants were collected for fluorescence measurement of porphyrin by spectrofluorometer to quantify the cell uptake of the porphyrin molecule. To further examine the fluorescence activation of USPV versus porphysome, confocal imaging was conducted to monitor the porphyrin fluorescence change with time after cell incubation. 5×10⁴ cells/well were seeded in eight-well chamber slides 24 h prior to incubation. Cells were incubated with porphysomes and USPV at porphyrin concentration of 10 μM for 3 h at 37° C., rinsed with PBS for 3 times and re-incubated in fresh cell culturing media. Cells were imaged by confocal microscopy (Olympus FluoView 1000, Laser 633 nm, Em at)) immediately and at 3 h, 6 h post medium change.

Evaluation of USPV as Theranostics for Glioma Tumor Treatment

Animal Preparation and Tumor Model

All animal experiments were performed in compliance with University Health Network guidelines. The animal studies were conducted on orthotopic 9L^{luc} gliosarcoma-, U87^{GFP} and U87^{luc} glioma-bearing nude mice. Nu/nu nude female mice were purchased from Harlan Laboratory and kept in the Animal Research Centre of University Health Network. To establish the models, animals will be anesthetized with an intraperitoneal injection of ketamine, xylazine and acepromazine (80 mg/kg, 5 mg/Kg, and 2.5 mg/kg), respectively. A 1 mm diameter burr hole will be made in the left hemisphere using a Dremel tool, exposing the dura but leaving it intact. 5×10⁴ of U87 cells or 1×10⁴ 9 L cells in 3 uL of media will be injected to the left hemisphere. Tumor size will be monitored weekly by magnetic resonance imaging (MRI). The experiments were conducted approximately 18 days post-inoculation when the tumors reached diameter of 4-5 mm.

Blood Clearance Study

USPV, PEG-USPV and folate-PEG_USPV were iintravenously injected to BALB/c mice at the dose of 2.5 mg/kg (n=4). Blood was collected from the leg vein of the mice serially prior to and after the injection (5 min, 30 min, 1 h, 2 h, 4 h, 8 h, 12 h, 24 h and 48 h). Blood were placed at room temperature for 30 min to separate plasma, and then centrifuged for 10 min at the rate of 12,000 rpm. The fluorescence of the supernatant was measured by Spectrofluorometer (HORIBA Scientific Inc.) to calculate the porphyrin amount in the blood (Excitation 420 nm, Emission, 675 nm, Slit width: 5 nm). The porphyrin amount at each time point was then analyzed by Graphpad Prism® to calculate half-life of the particles.

In Vivo and Ex Vivo Fluorescence Imaging

To study the specific tumor uptake and image-guided drug delivery capacity of USPV in vivo, fluorescence imaging was performed after the systematic administration. Tumor-bearing mice were fed with low-fluorescence diet (Harlan Tekland®, Product No. TD.97184) for 3 days before USPV administration. USPV-DiR-BOA were then injected through tail vein at a dose of 10 mg/kg on porphyrin content. Fluorescence images were acquired using a Maestro imaging system (CRI, USA) with a (575-605 nm excitation/680-750 nm emission filter for pyro signal and 725-755 nm excitation/780 nm long-pass emission filter for DiR-BOA signal. At 24 h post-injection, fluorescence imaging was performed in vivo with or without scalp and with cranium opened up. After sacrificing the animals, brains and major organs including heart, lung, liver, spleen, kidneys, adrenals and muscle were harvested and subjected to ex vivo fluorescence imaging. FMT (Fluorescence molecular tomography, PerkinElmer VisEn FMT 2500 LX Quantitative Tomography System, VisEn Medical Inc, Bedford, Mass.) imaging and in vivo confocal microscopic imaging (Leica FCM1000, Cellvizio® Technology, Ex: 660 nm, Em 689-900 nm) were also performed on tumor-bearing brains. For 9L^{luc}- and U87^{luc} glioma-bearing mice, luciferase solution was injected intraperitoneally 10 min before imaging. Bioluminescence imaging was also performed both in vivo and ex vivo.

Photodynamic Therapy

The PDT efficacy of USPV was investigated on U87^{GFP} tumor bearing mice. Four groups were included: blank control group without any treatment; PDT laser alone; USPV injection alone; USPV plus PDT laser treatment. When tumor reached 1 to 1.5 mm diameter, USPV were intravenously injected to animals at a dose of 5 mg/kg, calculated on the porphyrin content. At 24 h post-injection, mice were anesthetized with 2% (v/v) isoflurane and tumors were irradiated with a 671 nm laser (DPSS LaserGlow Technologies, Toronto, Canada). The laser intensity was measured as 50 mW/cm² with a spot size of 9 mm diameter and 3.5 mm in diameter as treatment area. Light doses of 37.5 J/cm² and 50 J/cm² were applied in the study. Temperature changes of tumors for the groups of laser alone and PDT treatment group were monitored using an infrared thermal camera (Mikroshot, LUMASENSE Technologies), and were calculated with n=5 in each treatment group for average and standard deviation.

Histological Analysis

To define the tumor margin, brains were frozen in liquid nitrogen after ex vivo fluorescence imaging and then cut into slides of 5 μm thickness using a Leica CM3050S cryostat. H&E staining was carried out by standard methods at the Pathology Research Program Laboratory at University Health Network. The sections were viewed and photographed by bright field microscopy at 20×. To evaluate the therapeutic efficacy, brains from each treatment group were harvested and fixed in 10% formaldehyde at 24 h post-treatment. H&E staining and TUNEL staining was carried out and subsequently analysed with the same standard protocols as above.

Tissue Slice Microscopic Imaging

The frozen slides were mounted with DAPI-containing mounting solution and imaged by Olympus FV1000 laser confocal scanning microscopy (Olympus, Tokyo, Japan) and Quorum WaveFX Spinning Disk Confocal (Yokogawa, Japan) with excitation wavelengths of 405 nm (DAPI channel), 491 nm (GFP channel) and 633 nm (Cy5.5 channel).

VX-2 Buccal Carcinoma Rabbit Model

The VX-2 buccal squamous cell carcinoma model was developed using the method described elsewhere (17, 18). Briefly, the tumor was harvested under sterile conditions from the freshly euthanized rabbit, placed in Hanks Balanced Salt Solution (HBSS, Sigma), washed twice with sterile HBSS, cut into small pieces, and stored at −80° C. until used. To obtain a single tumor cell suspension, the tumor pieces were thawed, minced and pressed through a 70 μm cell strainer. 300 μL of a high-density single cell suspension (˜5×10⁶/mL) are injected into the buccinators muscle (Buccal area) of an anaesthetized New Zealand white rabbit (2.8-3.3 kg).

Pharmacokinetic Study on HNC Rabbits

About 2 weeks after tumor induction when tumor size reached 1.5-2.0 cm, rabbits were intravenously injected with ⁶⁴Cu-USPV through a catheter in marginal ear vein (0.33 mg/kg for porphyrin, ˜5 mCi). Arterial blood was collected at 5 min, and 0.5, 1, 4, 8, 21, 30 h post-injection (n=4). The radioactivity of the plasma was determined as a function of concentration on a gamma-counter (Wizard 1480: PerkinElmer Inc., MA, USA). The clearance half-life was determined by log-linear regression.

PET/CT Imaging of HNC Rabbits

At 24 h post-injection of ⁶⁴Cu-USPV (0.33 mg/kg for porphyrin, ˜5 mCi), rabbits were anesthetized and subjected to PET imaging on MicroPET system (Focus 220: Siemens, Munich, Germany), and CT imaging on microCT system (Locus Ultra: GE Healthcare, U.K.) following 5 mL injections of Omnipaque 350 (GE Healthcare, Mississauga ON). PET/CT Images were registered and merged using Amira (FEI Visualization Sciences Group, Bordeaux, France). Volumes of interest were drawn on the merged CT images with Inveon Research Workplace (Siemens, Munich, Germany), and the standard uptake values (SUV) of ⁶⁴Cu-USPV were quantified from the registered images.

Biodistribution and Ex Vivo Fluorescence Imaging of USPV on HNC Rabbits

After PET/CT imaging the organs of rabbits including tumor, lymph node, salivary gland, lung, heart, liver, muscle, spleen, and kidneys were excised, weighed, and measured the radioactivity on a gamma-counter. Organ uptake was calculated as percentage of injected dose per percentage of total animal mass of the sample (SUV) for each rabbit. Ex vivo fluorescence imaging was performed with Maestro (Caliper Life Sciences, MA, U.S.A.) with yellow filter setting (excitation: 575-605 nm; emission: ≧0.645 nm detection, 200 ms exposure time).

Rabbit Tissue Pathology and Microscopic Imaging

Frozen tissue sections were fixed and treated with DAPI, H&E and Pan-Cytokeratin staining, respectively. High-resolution images of the stained sections were acquired on a scanning laser confocal microscope (TISSUEscope 4000, Huron Technologies).

Intraoperative Fluorescence Imaging

Real-time fluorescence-guided surgery on VX-2 rabbits was performed with an in-house fluorescence imaging endoscopy system (650±20 nm excitation, 700±25 nm emission) at 24 h after intravenous injection of 4 mg/kg of USPV. Guided with the fluorescence, tumor and suspicious lymph nodes were dissected until non-fluorescent nodules were left on the surgical bed of the animals.

PDT on HNC Rabbits

Four groups of VX-2 rabbits were included in the treatment study: blank control (n=3); PDT laser alone (n=3); USPV injection alone (n=3); USPV plus PDT laser treatment (n=4). When tumor size reached ˜300 mm³, USPV were intravenously injected to rabbits for USPV group and USPV-PDT group (4 mg/kg of porphyrin dose). For PDT treatment, rabbits were anesthetized and subjected to a two-step PDT procedure at 24 h post-injection. The first step was a straight laser irradiation (671 nm) on the exterior surface of the tumor with a light dose of 125 J/cm², laser power of 200 mW and irradiation area of 15 mm in diameter. Temperature changes of tumors during laser irradiation were monitored using the infrared thermal camera. The second treatment step involved the insertion of a fiber-optic cable (9 mm diffuse laser fiber) into the tumor to irradiate from the interior of the tumor with a light dose of 120 J/cm² and laser power of 100 mW. After the treatment, rabbits were put under standard protocol of care and the tumor growth was continuously monitored with microCT scanning. Terminal surgeries were performed on rabbits when the tumor size reached 5000 mm³. All four USPV-PDT rabbits were found tumor-free at about 30 days after treatment. They were euthanized at Day 34-36 post-PDT for further evaluation of treatment efficacy.

To evaluate the toxicity of the treatment, comprehensive biochemistry and haematology blood test of all treated rabbits were performed at 24 h post-injection, right before PDT, 1 week post- and 3 weeks-post-PDT treatment respectively. After terminal surgery, tissues from tumor region and other major organs were harvested at 24 h post-treatment, subjected to H&E and Pan-cytokeratin staining, and imaged with Aperio ImageScope to determine the remnant of malignancy. Two experienced pathologists evaluated all histopathology slides for malignancy identification and tumor eradication confirmation.

Statistical Analysis

The Student's t-test (two-tailed) was used to determine the statistical significance of the difference between different groups in TUNEL and toxicity study. P-values less than 0.05 were considered significance.

Results and Discussion

Synthesis and Characterization of USPV

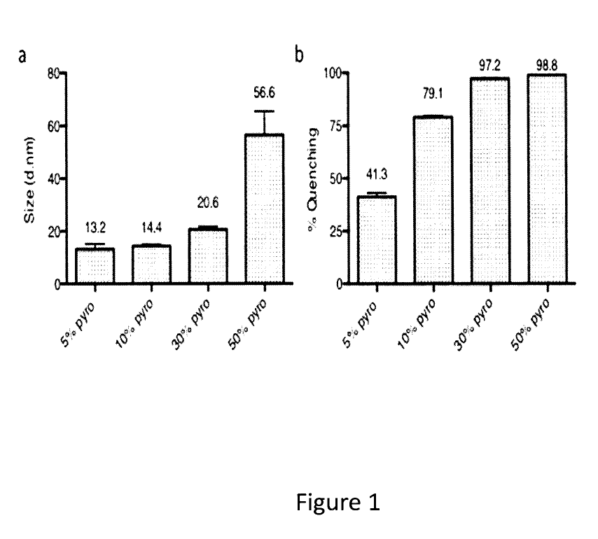

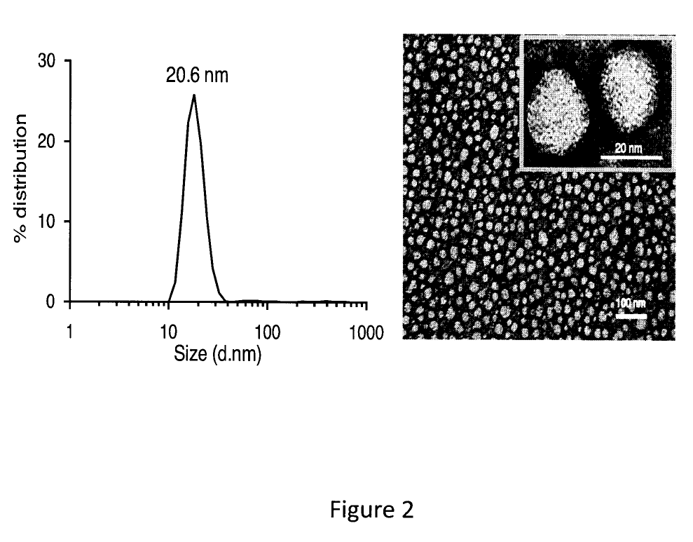

We created an ultra small size porphyrin vehicle (USPV) which has a hydrophobic core of cholesteryl oleate, enveloped by phospholipid monolayer of porphyrin lipid with DMPC, and constrained by an 18-amino acid ApoA-1 mimetic peptide. We found the structural and photophysical properties of the USPV are dependent on the ratio of porphyrin-lipid to DMPC. As shown in FIG. 1, increasing the ratio of porphyrin lipid to DMPC led to the enhanced porphyrin fluorescence quenching and increased particles size. When the ratio was over 30%, high fluorescence quenching (>95%) was achieved and the particles size was still controlled under 30 nm. The USPV with 30% mol porphyrin-lipid/70% mol of DMPC was chosen as an optimal USPV for further application studies, as it contained the maximum porphyrin lipid for a stable and monodisperse USPV, had favorable size (<30 nm, FIG. 2), and exhibited efficient fluorescence quenching.

The Absorption and Circular Dichroism (CD) Spectra of USPV

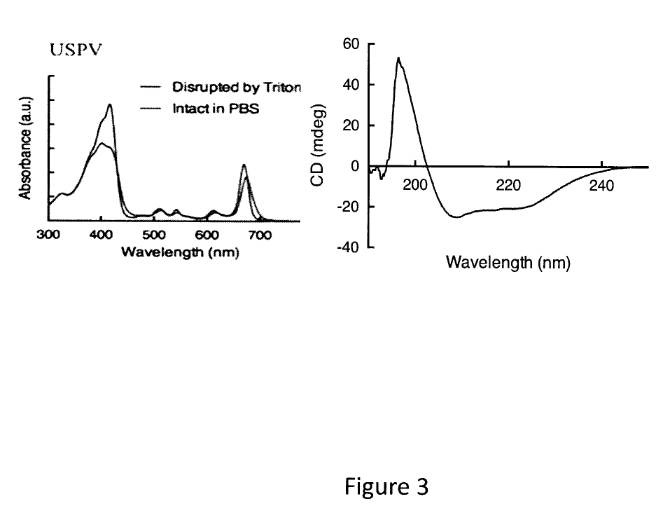

Based on the absorbance spectrum of pyropheophorbide-lipid (pyro-lipid), the estimated USPV extinction coefficient ε₆₈₀ was 7.8×10⁷ cm⁻¹M⁻¹. This enhanced light absorption indicates the high density of porphyrin environment in USPV. The CD spectrum confirmed the alpha helix structure of USPV (FIG. 3).

Fluorescence and Singlet Oxygen Generation

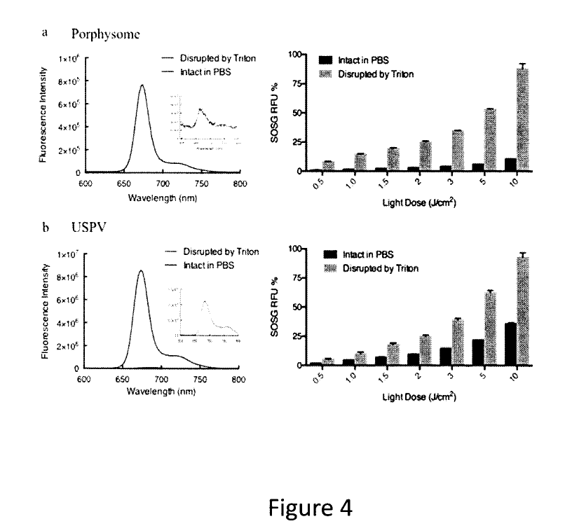

The optical properties of USPVs were investigated by comparing the fluorescence and singlet oxygen generation of the intact particles in PBS and its structure-disrupted samples in Triton X-100 at the same porphyrin concentration. As shown in FIG. 4, similar to that observed for porphysome, the high density of porphyrin environment extremely inhibited the fluorescence generation and the singlet oxygen production of USPV. The fluorescence of USPV was quenched by 100 fold when compared with the nanostructure-disrupted samples. Upon PDT laser (671 nm) irradiation at a wide range of light fluence (0.5-10 J/cm²), USPVs exhibited 2-3 fold less singlet oxygen generation when compared with the nanostructure-disrupted samples. Therefore, the intact USPV is photodynamic inactive, while it will become PDT active when the nanostructure is disrupted.

Cellular Uptake of USPV and In Vitro Fluorescence Activation

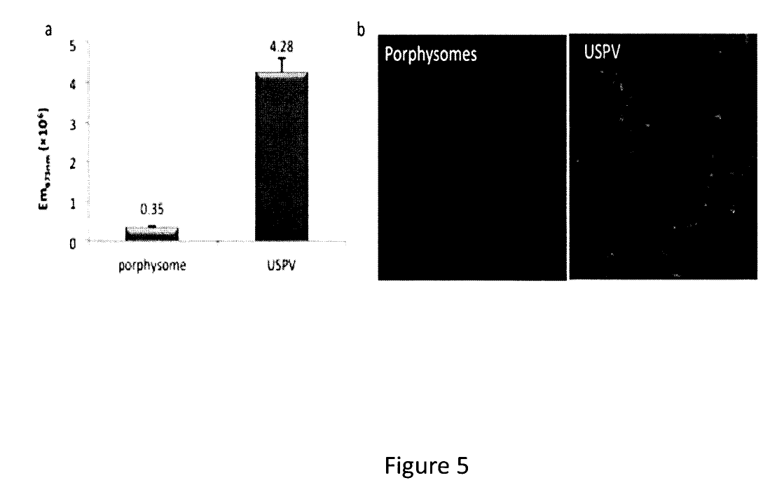

To investigate if the small-sized particle is favourable for intracellular uptake, the cellular uptakes of USPV and porphysomes were examined in U87 glioma cells by measuring the porphyrin fluorescence signals in cell lysis buffer. Compared to porphysomes, USPV showed about 10 times higher uptake in the U87 cell after incubation by the same concentration of porphyrin (FIG. 5a). The porphyrin fluorescence activation in cells was further assessed by confocal study. Unlike porphysome disruption in cells that is a time-consuming process, evidenced by the gradual unquenching of porphyrin fluorescence in cells, a strong porphyrin fluorescence was observed immediately in the U87 cells after 3 h incubation with USPV (FIG. 5b) and the fluorescence signal was not further enhanced significantly with time. Altogether, these data suggested that the small size USPV facilitated not only the cell internalization, but also the photoproperties activation in cells.

Blood Clearance

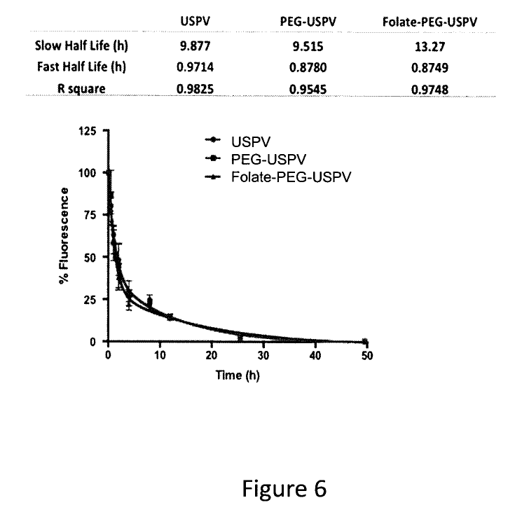

To examine the pharmacokinetics profile of USPVs, blood clearance study was performed on healthy mice. Three groups were included in the study: USPV, PEG-USPV (PEGylated USPV) and active targeting FR-USPV (folate receptor-targeted USPV). The porphyrin concentration in blood serum was measured at different time point post-administration using fluorescence measurement. As shown in FIG. 6, regardless of PEGylation, both USPV and PEG-USPV had similar and favorable circulation slow half-life (9.9 h for USPV and 9.5 h for USPV-PEG, respectively), indicating no need of PEGylation for improving in vivo circulation, whereas PEGylation is essential for most liposomal structures to ameliorate their stability in vivo. Interestingly, in contradictory to our previous observation that the involvement of folate-lipid in porphysome formulation shortened the particle in vivo circulation time,(10) FR-USPV exhibited a significantly prolonged slow half-life (13.3 h for folate-USPVs vs <4 h for FR-porphysomes). As EPR effect plays the key role in the tumor accumulation of nanoparticles, this prolonged circulation would benefit the infiltration of nanoparticles from the blood circulation directly into the tissues and enhance the retention of the particles in the targeting diseased area. Thus, more efficient targeting delivery and more effective photoproperties (fluorescence and singlet oxygen generation) activation would be expected for FR-USPV in FR-positive cancer types comparing to folate-porphysomes.

Tumor-Specific Accumulation of USPVs

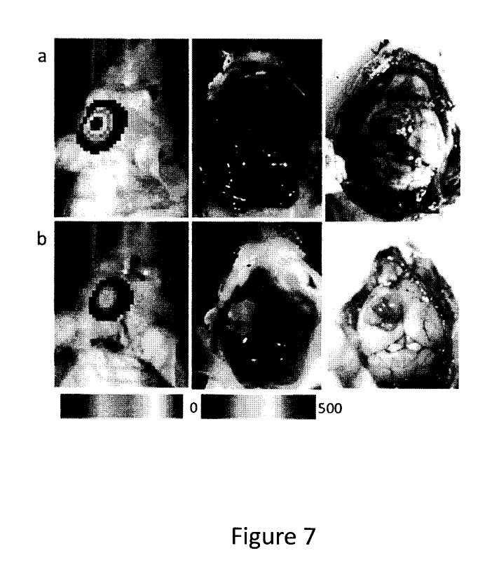

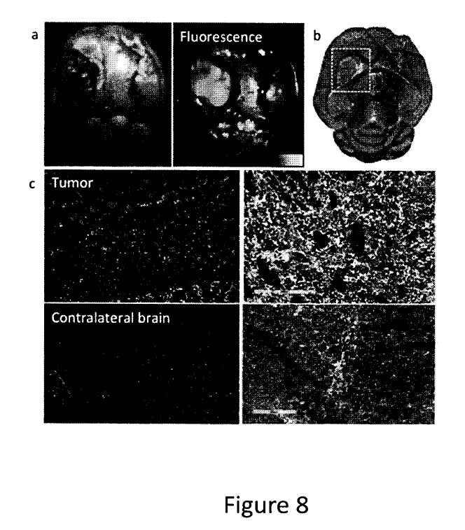

We recently developed a sub-40 nm porphyrin lipid nanodisc and demonstrated the small size nanodiscs displayed a 5-fold increase of diffusion coefficient in comparison to the larger size porphysomes (130 nm), in diffusing through a tumor's collagen-rich matrix.(19). Here we investigated the in vivo delivery advantage of small size USPV over porphysome. Mice with 9L^{luc} glioma were injected with USPV (21 nm) and porphysome (130 nm) at the porphyrin concentration of 200 nmol, and the mice crania were removed under anesthesia at 24 h post-administration to expose the tumors for fluorescence images in situ. As shown in FIG. 7, the middle column, both USPV and porphysome can delineate clearly the tumor from the surrounding healthy brain by fluorescence imaging which well-matched with the tumor sites defined by BLI imaging (FIG. 7, left column). However, the fluorescence signal from the USPV-administrated tumor was much stronger than that of the porphysome-dosed one, suggesting the benefit of the ultra small USPVs (<30 nm) on enhancing tumor-specific accumulation. The specificity of tumor accumulation of USPV in 9L^{luc} glioma tumor was further demonstrated by ex vivo brain tissue imaging (FIG. 8a), where the fluorescent core in brain marched well with the tumor region depicted by H&E histology slice (FIG. 8b). We further validated the tumor-specific uptake of USPV at microscopic level using confocal imaging of the frozen brain tissue slice, where strong porphyrin signal was observed only in tumor peripheral region, but not in contralateral brain area (FIG. 8c).

The Potential of USPV for Drug Delivery

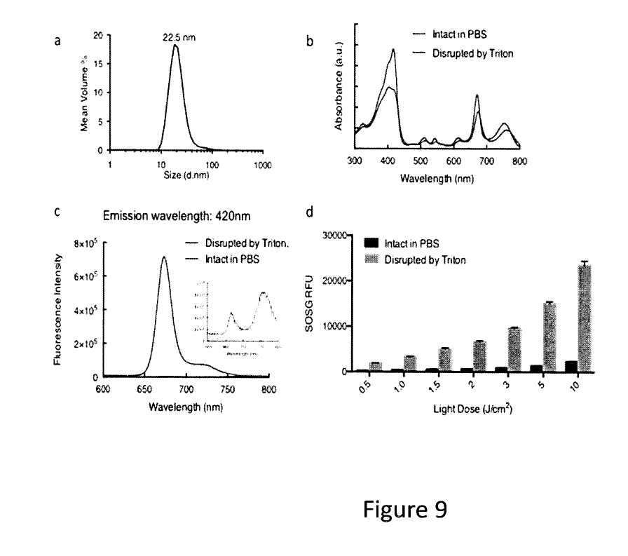

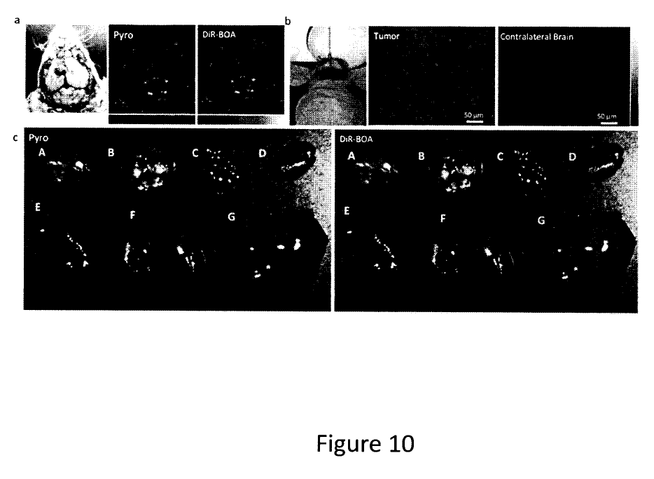

As USPV has a core-shell nanostructure with a hydrophobic core surrounded by lipid monolayer, it has amiable potential for loading and safe delivery of hydrophobic bioactive compounds. In this study, a near-infrared fluorescent hydrophobic dye, DiR-BOA, was used as a drug surrogate to examine the drug loading capacity and delivery behaviors of USPV. By adding 0.5 mol of DiR-BOA in the USPV formulation (0.9 μmol porphyrin-lipid, 2.1 μmol DMPC and 0.3 μmol CO), DiR-BOA was successfully loaded into the particle with loading efficiency of 85%. The resulted USPV(DiR-BOA) with size of 22.5 nm (FIG. 9a) was quite stable in PBS at 4° C., as minimal size change and negligible DiR-BOA leakage were observed over 30 days. We then investigated the in vivo behaviours of the USPV(DiR-BOA) in orthotopic U87 glioma bearing mice. The mice after 24 h injection of USPV(DiR-BOA) were subjected to the crania removal surgery under anesthesia, and fluorescence imaged at porphyrin channel (Ex: 615 nm, Em: 680-750 nm) and NIR drug surrogate channel (Ex: 750 nm, Em: 780-950), respectively, using CRI Maestro™ imaging system. As shown in FIG. 10a, both porphyrin and DiR-BOA signals were highly concentrated in the tumor, which clearly delineated tumor margin while sparing healthy brains close-by. In addition, these two fluorescence signals were well-colocalized, suggesting that the USPV(DiR-BOA) enable a stable and efficient delivery of drug surrogate selectively in tumor. More interestingly, this highly efficient delivery allowed for fluorescence detection of tumor cells at microscopic level by an in vivo fluorescence confocal microscopy with a deep-red long-pass filter, while sparing non-fluorescent contralateral brain cells (FIG. 10b). To further validate the tumor-specific accumulation of USPV, its tissue biodistribution was examined by fluorescence imaging when the animals were sacrificed. As shown in FIG. 10c, only glioma tumor and liver exhibited strong fluorescence signals of porphyrin and DiR-BOA, while other organs showed negligible fluorescence, demonstrating an extremely high tumor-specific uptake of USPV(DiR-BOA). Similar to most nanoparticle's delivery, the high liver uptake of USPV was probably due to their hepatobillary clearance. But unlike most nanoparticle's delivery including porphysomes, a much lower spleen uptake of USPV was probably benefited from its ultra small size that contributed to the ‘escape’ from filtering-out by the reticuloendothelial system. The well-correlation between the porphyrin fluorescence and DiR-BOA fluorescence in all of the detected tissues (FIG. 10c) further demonstrated the structural intact of USPV(DiR-BOA) in systemic delivery to accumulation in various tissues. Altogether, these data suggested that 1) the USPV provides a highly tumor selective and efficient drug delivery system for cancer therapy with minimal pre-leakage and off-target effect; 2) due to the stable delivery characters, the porphyrin signal of USPV, such as fluorescence, could be used for tracking drug delivery to guide the treatment planning.

The Intrinsic ⁶⁴Cu-Labelling of USPV for PET Imaging

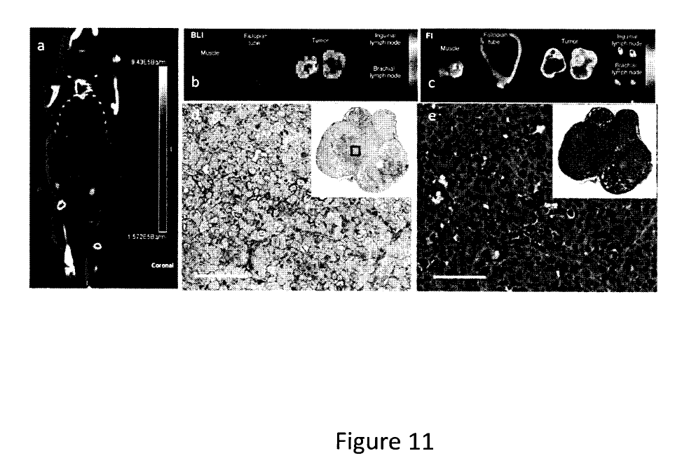

As porphyrins are great chelators for many metals, forming highly stable metallo-complex(20). Our previous study demonstrated the stable chelation of radioactive copper-64 (⁶⁴Cu) to the porphyrin-lipid of porphysomes, enabling PET imaging of in vivo fate of nanoparticle (11-12). Using a similar labelling approach, we successfully incorporated ⁶⁴Cu into the preformed USPV with high ⁶⁴Cu labelling efficiency (>95%) and followed by investigation of its delivery behaviors. As shown in FIG. 11, ⁶⁴Cu-USPV enabled selectively picking up ovarian cancer metastases, where metastases tumors exhibited super bright PET signal while the surrounding tissue, such as fallopian tube, showed minimal signal. The PET imaging-enabled tumor-specific picking up was further confirmed by ex vivo tissue porphyrin fluorescence imaging, which was well-correlated with the bioluminescence signal from tumor cells. The metastases tissue was further identified by histology analysis. Thus, the intrinsically ⁶⁴Cu labelling of USPV enable non-invasive and accurate tracking the nanoparticles delivery and additionally detect tumor due to its tumor-preferential accumulation, thus showing great promise for translation to clinical application.

In addition, USPV could be easily chelated with other metals. For example, the insertion of paramagnetic Mn³⁺ ion could generate contrast for MRI, (21) and incorporating palladium (Pd) in USPV could further improve singlet oxygen generation to maximize the PDT potency (22).

The Potential of USPV for Fluorescence-Guide Tumor Resection (FGR)

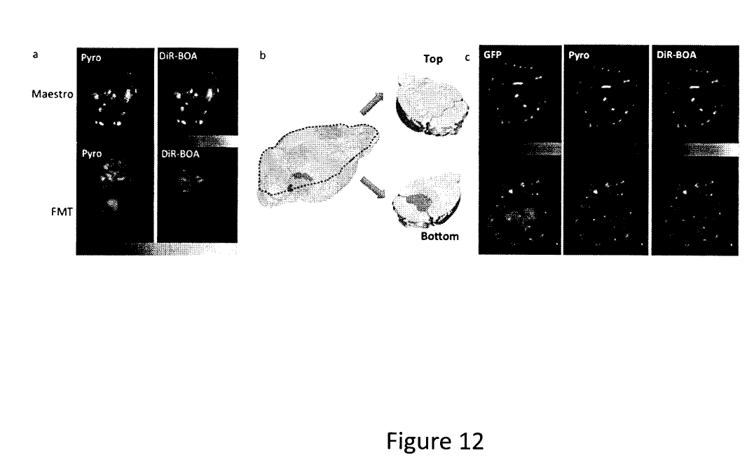

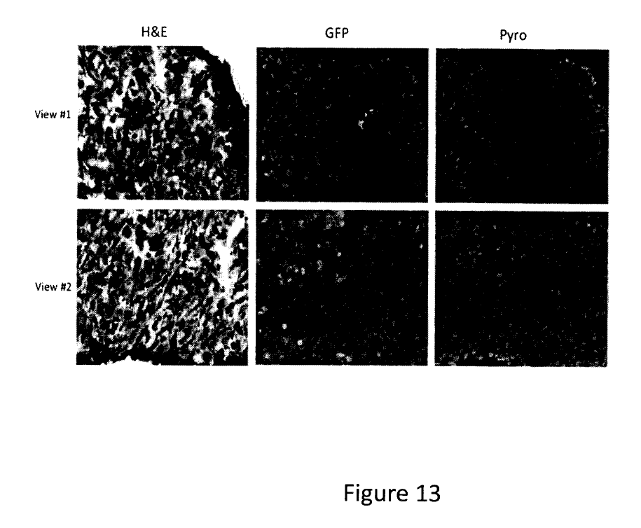

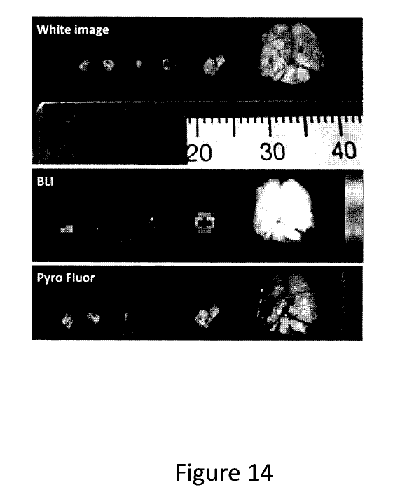

Surgical removal of the tumors remains still the mainstream of glioma treatment in clinical practice and the outcome is influential to the survival of the patients. The major challenge in the surgical procedure is to define positive margins. Insufficient surgery will result in the local recurrence of the tumor and the failure in salvage therapy, while over excision will lead to loss of important neuro functions. Thus the precise delineation of the cut-edge is essential for brain during surgery. We have demonstrated the capability of USPV(DiR-BOA) for visualizing tumor and delineating tumor region from surrounding health brain by the intrinsic porphyrin fluorescence and DiR-BOA signal at 24 h post systemic administration. We next investigate its potential application in fluorescence-guided glioma surgeries. To mimic the clinical scenario, an orthotopic U87^{GFP} glioma mouse model with tumor seeded deeply inside brain (5 mm from top surface) was utilized. As shown the FIG. 12a, after 24 h injection of USPV(DiR-BOA), neither intrinsic porphyrin fluorescence nor DiR-BOA fluorescence was observed from the top surface of intact brain using Maestro imaging system, but the both fluorescence signals could be detected clearly by FMT imaging. Following the transection process illustrated in FIG. 12b, the glioma tumor was exposed by removal the top part of brain. As the bottom part containing the solid tumor entity so the top part with minimal tumor residue was considered as a surgery bed. As shown in the FIG. 12c, both porphyrin and DiR-BOA signals were able to visualize and define tumor tissue accurately as they were well-correlated with the GFP fluorescence of tumor cells. The porphyrin fluorescent tissue was then collected and sent for histology analysis and frozen tissue slicing. As shown in FIG. 13, the H&E staining revealed the cancer cell morphology of the tissue. Meanwhile, the frozen tissue slide showed both GFP signal (from tumor cells) and porphyrin signal (from USPV) at microscopic level, further affirming the ability of USPV to depict tumor for imaging-guided surgery. Moreover, the porphyrin fluorescence of USPV, could identify multi-foci of U87^{luc} tumor that scattered through the mice brain ranging from 4 mm to less than 1 mm in size, even that could not been detected by MRI scanning. As shown in the FIG. 14, the removed fluorescent foci exhibited clearly the intrinsic bioluminescence signal of tumor cells. Taking together, these results demonstrated the high specificity and sensitivity of USPV for tumor identification, providing a good tool for fluorescence-guided glioma surgery.

The Potential of USPV as Activatable Photodynamic Nanobeacon

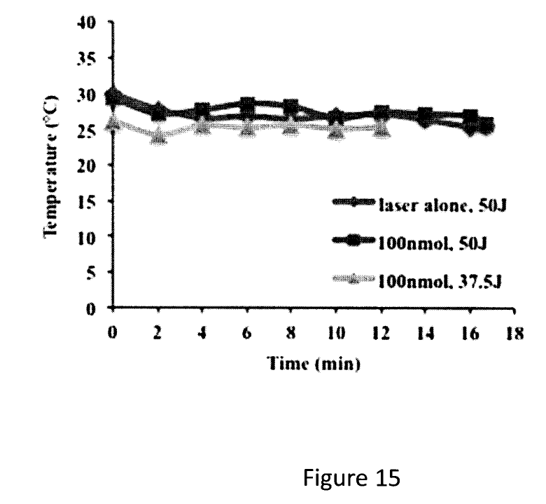

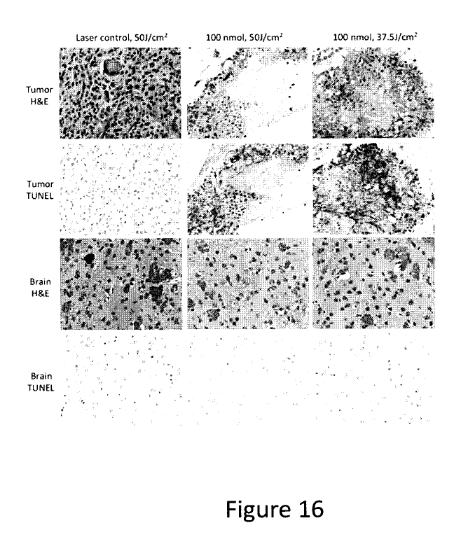

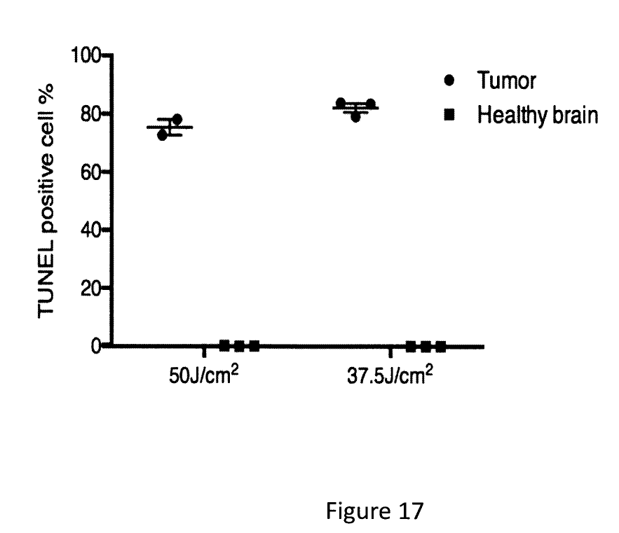

As both fluorescence and singlet oxygen generation of USPV are highly quenched in the intact nanostructure and could be quickly restored after accumulation in tumor. We investigated extensively the potential application of USPV for PDT in vivo. The fluorescence activation of USPV could serve as a useful indicator for assessment of the nanostructural disruption and singlet oxygen activation. As mentioned previously, glioma tumor displayed significant increase of porphyrin fluorescence at 24 h post-injection. We then chose this time point for laser irradiation. Briefly, the laser irradiation (671 nm, 50 mW/cm²) was applied trans-cranium through a small skin cut at light fluence of 50 J/cm² or 37.5 J/cm² after 24 h injection of USPV at porphyrin dose of 4 mg/kg. The tumor temperature during the laser irradiation was real-time monitored by a thermal camera. At 24 h post-treatment, animals were sacrificed and the brain tissues were prepared for histology analysis and TUNEL staining. The mice with glioma tumor receiving only laser irradiation and the mice with glioma tumor receiving USPV only were served as laser control and USPV control, respectively. No significant increase of tumor temperature (remained constantly around 27° C.) was observed for all laser treatment groups, indicating no photothermal effect contributed to the treatment (FIG. 15). The tumor tissue after USPV-PDT either at light fluence of 50 J/cm² or 37.5 J/cm² showed condensed nuclei and loss of cell structure in H&E staining, while the tumor tissue from USPV and laser controls remained unaffected (FIG. 16), indicating the USPV-enabled effective PDT and the noninvasiveness of USPV and laser irradiation alone. The TUNEL staining further confirmed that the USPV-PDT induced obvious cell apoptosis with 75.4% TUNEL-positive cells for 50 J/cm² group and 82.1% TUNEL-positive for 37.5 J/cm² group (FIG. 17), while non significant cell apoptosis was observed for control groups. In addition, no observable histology change and apoptosis in surrounding brain tissue of USPV-PDT group, indicating the negligible side effect caused by USPV-PDT. Therefore, USPV enable tumor-specific PDT at very low light dose while preservation of normal health, thus providing a safe PDT treatment protocol.

The Pre-Clinical Application of USPV for Head-and-Neck Cancer (HNC) Management in a Large Animal Rabbit Model.

The low survival rate of HNC patients is attributable to late disease diagnosis and high recurrence rate. The current HNC staging suffer from inadequate accuracy and low sensitivity of diagnosis for appropriate treatment management. The USPV with intrinsic multimodalities of PET, fluorescence imaging, and PDT might provide great potential to enhance the accuracy of HNC staging and revolutionize HNC management. Using a clinical relevant VX-2 buccal carcinoma rabbit model which could consistently develop metastasis to regional lymph nodes after tumor induction, we investigated the abilities of USPV for HNC diagnosis and management.

USPV-PET Enabled Detection of Primary Tumor and Sentinel Lymph Nodes in HNC Rabbit Model

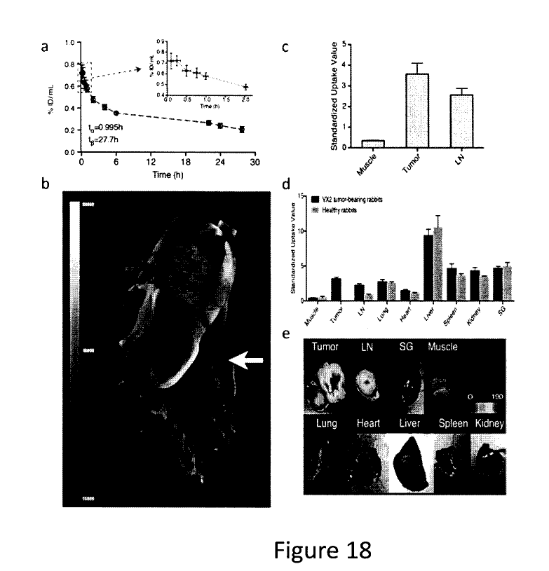

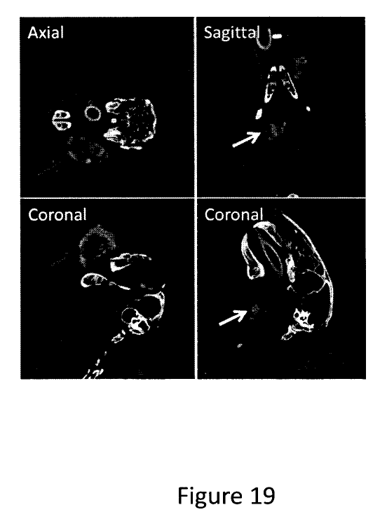

The blood clearance profile of ⁶⁴Cu-USPV in VX-2 rabbit was fitted to a two-compartment model, showing a favorably slow half-life up to 27.7 h (FIG. 18a). Therefore, PET imaging was performed on VX2 rabbits at 24 h post intravenous injection of ⁶⁴Cu-USPV (0.34 mg/kg of porphyrin, ˜5 mCi) to match its biological half-life and radionuclide half-life (⁶⁴Cu t½=12.7 h). As shown in the PET/CT co-registered image (FIG. 18b, FIG. 19), the tumor and sentinel lymph node (SLN) were clearly distinguishable with high contrast. Consistent with the rendered image, tumor and SLN showed significantly higher standard uptake values (SUV) quantified from PET volume-of-interest (VOI) measurements comparing to that of surrounding muscle, which were 3.58±0.53, 2.57±0.53 and 0.35±0.02 respectively (n=5, P<0.05, FIG. 18c).

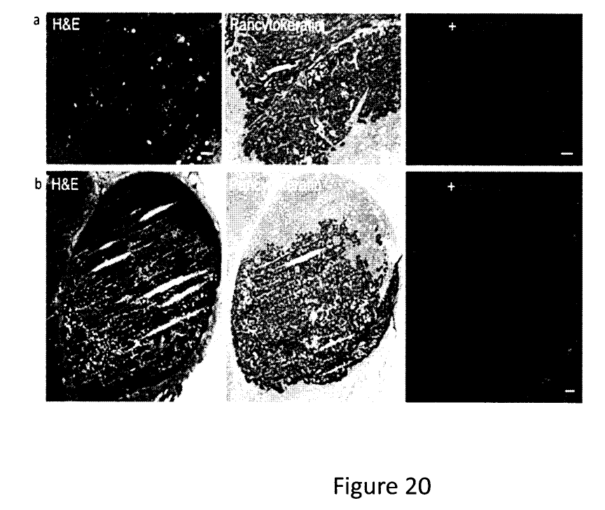

The distribution of ⁶⁴Cu-USPVs in major organs was further evaluated by gamma-counting method, which revealed similar distribution patterns of USPV in tumor-bearing and healthy rabbits (FIG. 18d). The relatively higher standard uptake value (SUV) of liver (9.34±0.92 SUV and 10.54±1.68 SUV for tumor-bearing and healthy rabbits, respectively) was likely due to hepatobiliary clearance of ⁶⁴Cu-USPVs. However, this high uptake would not affect HNC detection considering the relative remote location of liver from head and neck region. The average uptake of tumor and SLN from gamma-counting were 3.14±0.26 SUV and 2.21±0.26 SUV respectively (FIG. 18d, n=5), which are consistent to their corresponding SUVs got from PET image VOI quantification (FIG. 18c). The SLN of tumor-bearing rabbits exhibited significantly higher uptake than that of healthy rabbits (0.87±0.13 SUV, n=3, P<0.01) is likely due to the elevated lymphatic flow and the presence of metastatic lesions that were identified by H&E analysis and Pan-Cytokeratin (PanCK) staining (FIG. 20). Therefore, 64Cu-USPVs were capable of delineating malignant SLNs from healthy ones.

The following ex vivo fluorescence imaging of the resected tissues further confirmed the significantly higher accumulation and fluorescence activation of USPVs in tumor and draining SLN of tumor-bearing rabbits (FIG. 18e). Negligible fluorescence signal was observed in the salivary glands in spite of the relatively high accumulation of ⁶⁴Cu-USPVs (FIG. 18e), likely due to the fact that though USPV non-specifically accumulated in salivary glands like other PET image agents (e.g. ¹⁸F-FDG), it remained intact and non-fluorescent. These results indicated that by engaging PET and fluorescence imaging, USPV was able to provide complementary information for accurate detection of metastatic lymph nodes and potentially could be employed for image-guided resection of lymph node with low background fluorescence of the salivary gland.

Fluorescence-Guided Resection of Primary Tumor and Metastatic Disease

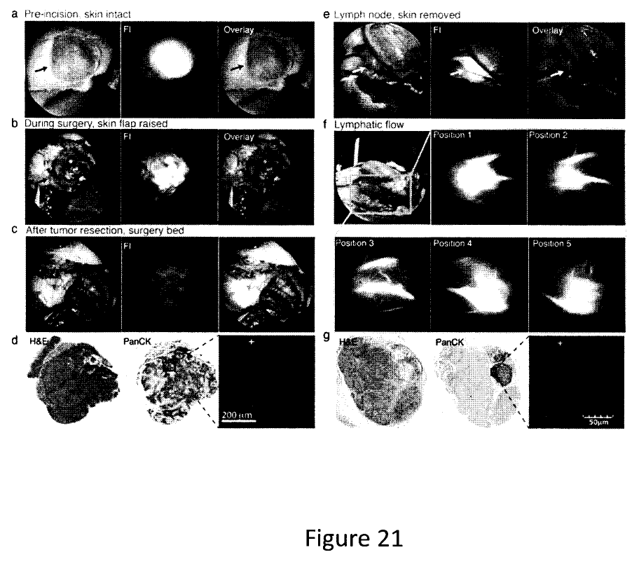

By taking advantage of selective fluorescence activation of USPVs in tumor and metastatic lymph node(s), we evaluated the capacity of USPVs as fluorescent intraoperative guidance for surgical resection of primary tumors and SLN(s) in tumor-bearing rabbits. As shown in FIG. 21a, the tumor (with skin intact) was sufficiently fluorescent for visualization compared to surrounding tissue under an in vivo fluorescence imaging system. Upon raising the skin flap during surgical exploration, the tumor was exposed and was clearly depicted by porphyrin fluorescence (FIG. 21b). Guided by the fluorescence, all suspicious malignancies around the check were surgically removed. The surgery bed exhibited negligible fluorescence signal, suggesting the complete tumor resection (FIG. 21c). The resected tissues were confirmed to be malignant by histological analysis (FIG. 21d). The porphyrin fluorescence in the tissue histology slides was corresponded well with cancer cell morphology and positive PanCK staining, indicating that USPV fluorescence highlighted the primary tumor with considerable specificity and accuracy at cellular level (FIG. 21d). Likewise, USPV fluorescence also delineated the draining SLN in vivo (FIG. 21e). Notably, the lymphatic network from primary tumor to SLN, and to regional lymph nodes was exquisitely mapped by the fluorescence signal (FIG. 21f). Following the orientation of the lymphatic network (zoomed-in images, positions 1-5 in FIG. 21f), the secondary positive lymph node and the lymphatic spread pattern was identified. Histology studies affirmed the metastasis in the lymph node and strong porphyrin fluorescence was observed in the PanCK-positive area, suggesting the uptake of USPV in the metastatic region (FIG. 21g). Altogether, USPV fluorescence not only clearly delineates the primary tumor and malignant lymph node(s), but also the regional lymphatic network, which may potentially aid in nodal staging of HNC patients and reveal malignant lymph nodes prior to resection and pathological analysis.

USPV-Enabled PDT Induced Apoptosis

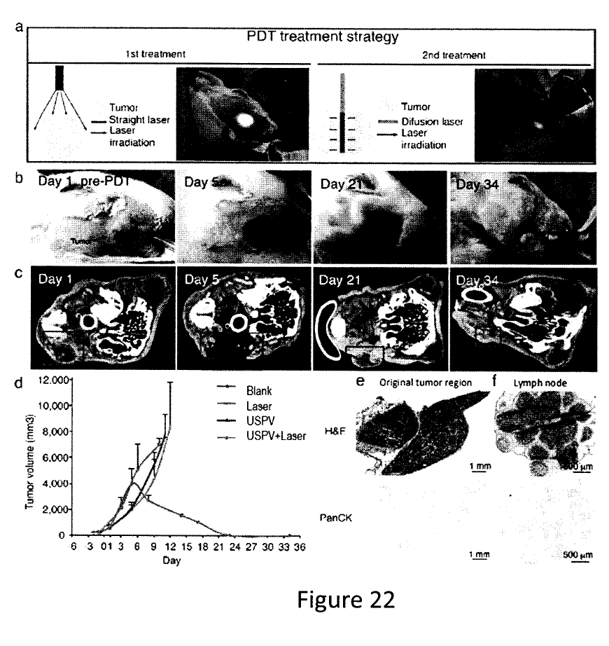

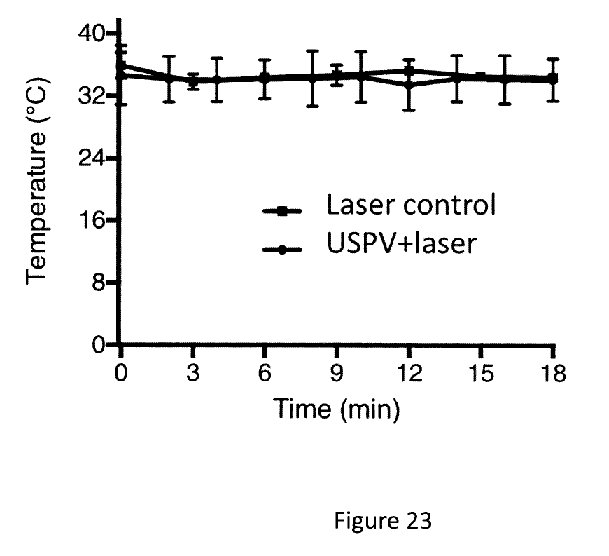

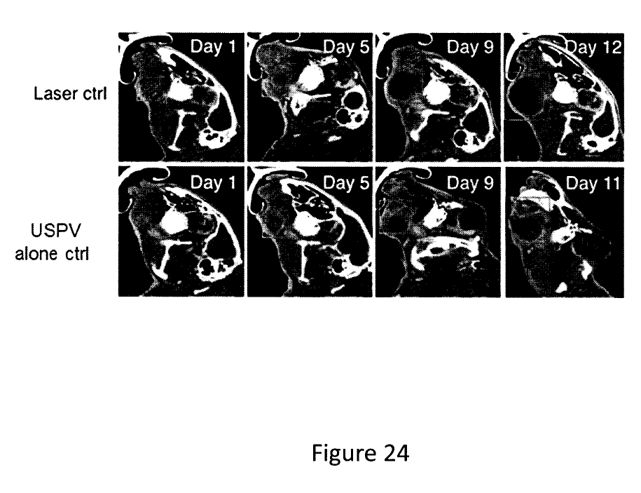

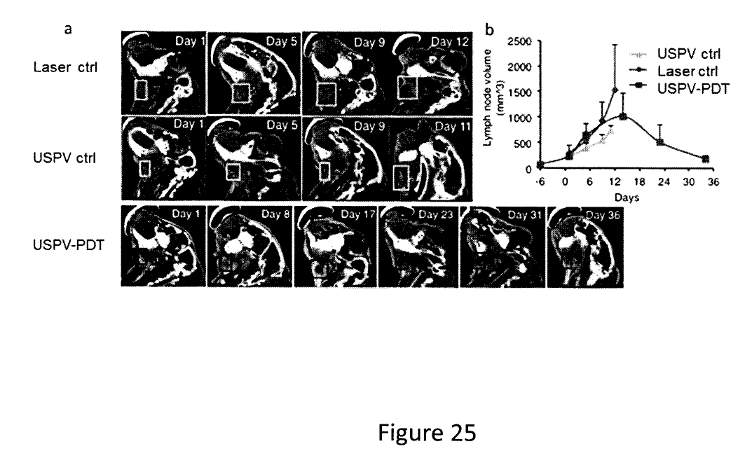

The long-term therapeutic effect of USPV-PDT was assessed on HNC rabbits. Tumor-bearing rabbits with average tumor sizes of 300 mm³ were categorized into four groups, including blank control (n=3), laser only control (n=3), USPV only control (n=3) and USPV-PDT group (n=4). As shown in FIG. 22a, a two-step laser irradiation strategy was used for the PDT at 24 h post-USPV injection in order to irradiate the entire tumor area. The absence of significant temperature increase during the laser treatment confirmed no thermal effect of the treatment, excluding the concern that thermal effect may cause unintended side effects on neighbouring health tissues (FIG. 23). USPV-PDT caused scarring around the tumor beginning from 24 h post-PDT, until 26 days post-treatment. Ultimately, all USPV-PDT rabbits were with no palpable tumor at day 34 post-treatment (FIG. 22b). Post-treatment tumor volumes were quantitatively determined by the volumetric measurement of 3D reconstructed microCT images. The USPV-PDT group showed a slight tumor size increase within the first week post-treatment, which was likely attributed to an expected inflammatory response and edema caused by PDT (FIG. 22c). However, the tumor size gradually declined from 6 days post-PDT until no tumor was detected at the day 34 post-PDT. In contrast, the control groups that received either the laser irradiation or USPV administration alone showed exponential tumor growth, similar to the blank control, indicating that neither of them induced any therapeutic effects (FIG. 22d, FIG. 24). The control groups reached the end point (tumor volume>5000 mm³) at day 6 for blank control, day 8 for laser control, and day 9 for USPV control (FIG. 5d), respectively. USPV-PDT enabled complete tumor ablation was further affirmed by pathological analysis, which demonstrated that the tissues resected from the original tumor area at terminal surgery did not exhibit pathological cell morphology, in addition to its negative PanCK staining (FIG. 22e). Notably, although have not received a direct laser irradiation, the lymph nodes of USPV-PDT group showed a gradual decrease in size from 14 days post-PDT (FIG. 25). All lymph nodes from the USPV-PDT group were found metastasis-free at 34 days post-PDT evidenced by pathology and PanCK staining analysis (FIG. 22f). These results strongly suggest that for HNC subtypes that are surgically inaccessible or adjacent to critical anatomical structures, such as the oropharynx, nasopharynx, hypopharynx and for recurrence cases, USPV-PDT may serve as an alternative approach to radiation treatment and chemotherapy to increase therapeutic efficacy and decrease long-term toxicity. USPV-PDT appears to be exceedingly effective, highly localized, and allows for the preservation of healthy tissue function.

USPV is a Safe Multi-Functional Nanoplatform

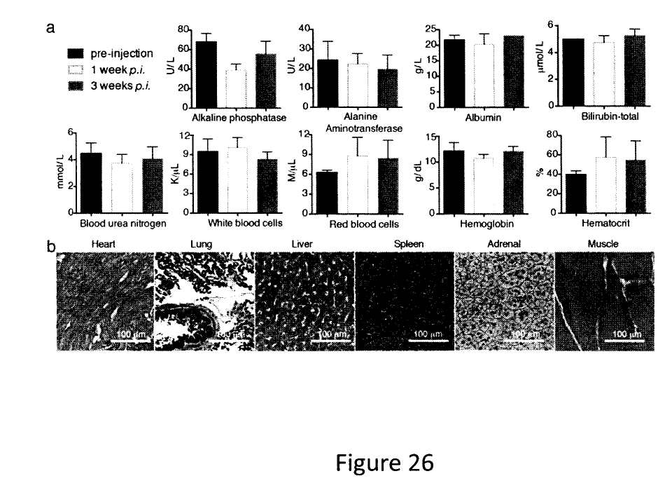

The toxicity of USPV-PDT to rabbits was assessed by blood tests periodically (FIG. 26a). The hepatic function of rabbits after treatment maintained normal with no significant changes, except for alkaline phosphatase (ALP), which showed moderate decrease within the normal range (from 68.1±8.66 to 43.5±9.67 U/L) at 1 week after treatment and returned to the baseline level over time (normal range 12-98 U/L). Red blood cell level remained stable after treatment, indicating that no interference with the physiological regulation of endogenous porphyrin (heme). White blood cell counts also remained unaffected, suggesting that no immunogenic effects were caused by USPV. Post-mortem histology analysis on USPV-PDT rabbits did not show abnormal cellular morphology in the heart, lung, liver, spleen, adrenal or muscle (FIG. 26b). These results suggest that USPV-enabled PDT treatment is a safe therapeutic approach.

In summary, there is described herein a multimodal theranostic porphyrin vehicle with a hydrophobic core, enveloped by porphyrin lipid based phospholipid monolayer, and constricted by an alpha helix structure. The porphyrins which high densely packed in intact USPV caused significant quenching of their photoactivities, including fluorescence and singlet oxygen generation, while become photodynamic active when the nanostructure is disrupted. The USPV has many favorable features for drug delivery such as hydrophobic drug-loading capability, ultra small size (<30 nm), and excellent blood circulation characteristics (10 h circulation half-life in mouse, 27 h in rabbit) with no need of PEGylation. We validated USPV being a stable drug delivery platform for tumor-specific delivery. The intrinsic ⁶⁴Cu labeling of USPV enabled non-invasive tracking of drug delivery, thus providing a useful mean for rational dosimetry and treatment planning. In a clinic relevant lymphatic metastases rabbit model, we demonstrated that USPV facilitated accurate detection of primary tumor and metastatic nodes, and enabled visualizing the lymphatic drainage from tumor to regional lymph nodes by both pre-operative PET and intra-operative fluorescence imaging. The insight of metastatic lymphatic pathways might permit the identification of unknown primaries and recurrent tumors with greater sensitivity to improve therapeutic outcome. Moreover, the effective photoproperties activation of the high densely packed porphyrins following tumor accumulation allowed for a precise fluorescence-guided tumor resection and a potent PDT in both glioma mouse and HNC rabbit model to afford complete eradication of primary tumors and blockage of tumor metastasis without damage of adjacent critical structures. Thus, the intrinsic multimodal nature and favorable delivery features of USPV confers high potential for cancer theranostics and clinical translation to enhance cancer diagnosis by integrating PET/CT and fluorescence imaging, and improve cancer therapeutic efficacy and specificity by tailoring treatment via fluorescence-guided surgical along with selective PDT.

Although preferred embodiments of the invention have been described herein, it will be understood by those skilled in the art that variations may be made thereto without departing from the spirit of the invention or the scope of the appended claims. All documents disclosed herein, including those in the following reference list, are incorporated by reference.

### REFERENCE LIST

1. Dougherty T J, et al. (1998) Photodynamic therapy. (Translated from eng) J Natl Cancer Inst 90(12):889-905 (in eng).
2. Ethirajan M, Chen Y, Joshi P, & Pandey R K (2011) The role of porphyrin chemistry in tumor imaging and photodynamic therapy. (Translated from eng) Chem Soc Rev 40(1):340-362 (in eng).
3. Stefflova K, Chen J, & Zheng G (2007) Killer beacons for combined cancer imaging and therapy. (Translated from eng) Curr Med Chem 14(20):2110-2125 (in eng).
4. Zheng G, et al. (2007) Photodynamic molecular beacon as an activatable photosensitizer based on protease-controlled singlet oxygen quenching and activation. (Translated from eng) Proc Natl Acad Sci USA 104(21):8989-8994 (in eng).
5. Josefsen L B & Boyle R W (2012) Unique diagnostic and therapeutic roles of porphyrins and phthalocyanines in photodynamic therapy, imaging and theranostics. (Translated from eng) Theranostics 2(9):916-966 (in eng).
6. Chen B, Pogue B W, & Hasan T (2005) Liposomal delivery of photosensitising agents. (Translated from eng) Expert Opin Drug Deliv 2(3):477-487 (in eng).
7. Komatsu T, Moritake M, Nakagawa A, & Tsuchida E (2002) Self-organized lipid-porphyrin bilayer membranes in vesicular form: nanostructure, photophysical properties, and dioxygen coordination. (Translated from eng) Chemistry 8(23):5469-5480 (in eng).
8. Ghoroghchian P P, et al. (2005) Near-infrared-emissive polymersomes: self-assembled soft matter for in vivo optical imaging. (Translated from eng) Proc Natl Acad Sci USA 102(8):2922-2927 (in eng).
9. Lovell J F, et al. (2011) Porphysome nanovesicles generated by porphyrin bilayers for use as multimodal biophotonic contrast agents. (Translated from eng) Nat Mater 10(4):324-332 (in eng).
10. Jin S C, Cui L, Wang F, Chen J, & Zheng G (2014) Targeting-triggered porphysome nanostructure disruption for activatable photodynamic therapy. Adv. Healthcare Mater.
11. Liu T W, MacDonald T D, Shi J, Wilson B C, & Zheng G (2012) Intrinsically copper-64-labeled organic nanoparticles as radiotracers. (Translated from eng) Angew Chem Int Ed Engl 51(52):13128-13131 (in eng).
12. Liu T W, et al. (2013) Inherently multimodal nanoparticle-driven tracking and real-time delineation of orthotopic prostate tumors and micrometastases. (Translated from eng) ACS Nano 7(5):4221-4232 (in eng).
13. Pluen A, et al. (2001) Role of tumor-host interactions in interstitial diffusion of macromolecules: cranial vs. subcutaneous tumors. (Translated from eng) Proc Natl Acad Sci USA 98(8):4628-4633 (in eng).
14. Cabral H, et al. (2011) Accumulation of sub-100 nm polymeric micelles in poorly permeable tumours depends on size. (Translated from eng) Nat Nanotechnol 6(12):815-823 (in eng).
15. Sykes E A, Chen J, Zheng G, & Chan W C (2014) Investigating the Impact of Nanoparticle Size on Active and Passive Tumor Targeting Efficiency. (Translated from Eng) ACS Nano (in Eng).
16. Corbin R I, et al. (2007) Enhanced Cancer-Targeted Delivery Using Engineered High-Density Lipoprotein-Based Nanocarriers J. Biomed. Nanotechnol. 3:367-376.
17. Li S J, Ren G X, Jin W L, and Guo W. Establishment and characterization of a rabbit oral squamous cell carcinoma cell line as a model for in vivo studies. Oral Oncol. 2011; 47(1):39-44.
18. Lin L M, Chen Y K, Chen C H, Chen Y W, Huang A H, and Wang W C. VX2-induced rabbit buccal carcinoma: a potential cancer model for human buccal mucosa squamous cell carcinoma. Oral Oncol. 2009; 45(11):e196-203.
19. Ng K K, Lovell J F, Vedadi A, Hajian T, & Zheng G (2013) Self-assembled porphyrin nanodiscs with structure-dependent activation for phototherapy and photodiagnostic applications. (Translated from eng) ACS Nano 7(4):3484-3490 (in eng).
20. Ali H & van Lier J E (1999) Metal complexes as photo- and radiosensitizers. (Translated from eng) Chem Rev 99(9):2379-2450 (in eng).
21. MacDonald D T, Liu W T, & Zheng G (2014) An MRI-Sensitive, Non-Photobleachable Porphysome Photothermal Agent. Angewandte Chemie, Int. Ed., 53.
22. Roxin A, Chen J, Paton A S, Bender T P, & Zheng G (2014) Modulation of reactive oxygen species photogeneration of bacteriopheophorbide a derivatives by exocyclic E-ring opening and charge modifications. (Translated from eng) J Med Chem 57(1):223-237 (in eng).

1. A nanovesicle comprising a monolayer of phospholipid, porphyrin-phospholipid conjugate and a peptide encapsulating a hydrophobic core, wherein
the peptide comprises an amino acid sequence capable of forming at least one amphipathic α-helix;
the porphyrin-phospholipid conjugate comprises one porphyrin, porphyrin derivative or porphyrin analog covalently attached to a lipid side chain, preferably at the sn-1 or the sn-2 position, of one phospholipid;
the molar % of porphyrin-phospholipid conjugate to phospholipid is 35% or less;
the nanovesicle is 35 nm in diameter or less.

2. The nanovesicle of claim 1, wherein the molar % of porphyrin-phospholipid conjugate to phospholipid is 35% or less, 30% or less, 25% or less, or 20-30%.

3. The nanovesicle of claim 1, wherein the nanovesicle is substantially spherical and 30 nm in diameter or less, 25 nm in diameter or less, between 20-30 nm in diameter or about 25 nm in diameter.

4. The nanovesicle of claim 1 wherein the porphyrin, porphyrin derivative or porphyrin analog in the porphyrin-phospholipid conjugate is selected from the group consisting of hematoporphyrin, protoporphyrin, tetraphenylporphyrin, a pyropheophorbide, a bacteriochlorophyll, chlorophyll a, a benzoporphyrin derivative, a tetrahydroxyphenyl chlorin, a purpurin, a benzochlorin, a naphthochlorins, a verdin, a rhodin, a keto chlorin, an azachlorin, a bacteriochlorin, a tolyporphyrin, a benzobacteriochlorin, an expanded porphyrin and a porphyrin isomer.

5. The nanovesicle of claim 4, wherein the expanded porphyrin is a texaphyrin, a sapphyrin or a hexaphyrin and the porphyrin isomer is a porphycene, an inverted porphyrin, a phthalocyanine, or a naphthalocyanine.

6. The nanovesicle of claim 1 wherein the porphyrin in the porphyrin-phospholipid conjugate is pyropheophorbide-a acid.

7. The nanovesicle of claim 1 wherein the porphyrin in the porphyrin-phospholipid conjugate is a bacteriochlorophyll derivate.

8. The nanovesicle of claim 1 wherein the phospholipid in the porphyrin-phospholipid conjugate comprises phosphatidylcholine, phosphatidylethanoloamine, phosphatidylserine or phosphatidylinositol.

9. The nanovesicle of claim 8, wherein the phospholipid comprises an acyl side chain of 12 to 22 carbons.

10. The nanovesicle of claim 1 wherein the phospholipid in the porphyrin-phospholipid conjugate is 1-Palmitoyl-2-Hydroxy-sn-Glycero-3-Phosphocholine or 1-Stearoyl-2-Hydroxy-sn-Glycero-3-Phosphocholine.

11. The nanovesicle of claim 1 wherein the porphyrin-phospholipid conjugate is pyro-lipid.

12. The nanovesicle of claim 1 wherein the porphyrin-phospholipid conjugate is oxy-bacteriochlorophyll-lipid.

13. The nanovesicle of claim 1 wherein the porphyrin is conjugated to the glycerol group on the phospholipid by a carbon chain linker of 0 to 20 carbons.

14. The nanovesicle of claim 1, wherein the porphyrin-phospholipid conjugate comprises a metal chelated therein, optionally a radioisotope of a metal.

15. The nanovesicle of claim 14 wherein the metal is selected from the group consisting of Zn, Cu, Mn, Fe and Pd.

16. The nanovesicle of claim 1, wherein the phospholipid is an anionic phospholipid.

17. The nanovesicle of claim 16, wherein the phospholipid is selected from the group consisting of phosphatidylcholines, phosphatidylethanolamines, phosphatidic acid, phosphatidylglycerols and combinations thereof.

18. The nanovesicle of claim 16, wherein the phospholipid is selected from the group consisting of 1,2-dipalmitoyl-sn-glycero-3-phosphatidic acid (DPPA), 1,2-dipalmitoyl-sn-glycero-3-phosphatidylcholine (DPPC), 1,2-distearoyl-sn-glycero-3-phosphocholine (DSPC), 1,2-dimyristoyl-sn-glycero-3-phosphocholine (DMPC), 1,2-dibehenoyl-sn-glycero-3-phosphocholine (DBPC), 1,2-diarachidoyl-sn-glycero-3-phosphatidylcholine (DAPC), 1,2-dilignoceroyl-sn-glycero-3-phosphatidylcholine (DLgPC), 1,2-dipalmitoyl-sn-glycero-3-[phosphor-rac-(1-glycerol)] (DPPG) and combinations thereof.

19. The nanovesicle of claim 1, wherein the peptide is selected from the group consisting of Class A, H, L and M amphipathic α-helices, fragments thereof, and peptides comprising a reversed peptide sequence of said Class A, H, L and M amphipathic α-helices or fragments thereof.

20. The nanovesicle of claim 19, wherein the peptide consists of consecutive amino acids of an apoprotein, preferably selected from the group consisting of apoB-100, apoB-48, apoC, apoE and apoA

21. The nanovesicle of claim 19, wherein the peptide is selected from the group consisting of 2F (DWLKAFYDKVAEKLKEAF)(SEQ ID NO. 1), 4F (DWFKAFYDKVAEKFKEAF)(SEQ ID NO. 2), and the reverse sequences of the foregoing

22. The nanovesicle of claim 19, wherein the peptide is the R4F peptide (Ac-FAEKFKEAVKDYFAKFWD)(SEQ ID NO. 3).

23. The nanovesicle of claim 20, wherein the at least one amphipathic α-helix or peptide is between 6 and 30 amino acids in length, 8 and 28 amino acids in length, 10 and 24 amino acids in length, 11 and 22 amino acids in length, 14 and 21 amino acids in length. 16 and 20 amino acids in length or 18 amino acids in length.

24. The nanovesicle of claim 1, wherein the hydrophobic core comprises a hydrophobic diagnostic or therapeutic agent.

25. The nanovesicle of claim 24, wherein the hydrophobic core comprises paclitaxel, docetaxel, 1,1′-dioctadecyl-3,3,3′,3′-tetramethylindotricarbocyanine iodide bis-oleate (DiR-BOA).

26. The nanovesicle of claim 1, wherein the nanovesicle is PEG free.

27. The nanovesicle of claim 1, further comprising PEG, preferably PEG-lipid, further preferably PEG-DSPE.

28. The nanovesicle of claim 1, further comprising a targeting molecule.

29. (canceled)

30. (canceled)

31. (canceled)

32. A method of performing photodynamic on a target area in a subject comprising:
a. providing the nanovesicle of claim 1;
b. administering the nanovesicle to the subject; and
c. irradiating the nanovesicle at the target area with a wavelength of light, wherein the wavelength of light activates the porphyrin-phospholipid conjugate to generate singlet oxygen.

33. The method of claim 32, wherein the target area is a tumor.

34. A method of delivering a hydrophobic agent to a subject comprising:
a. providing the nanovesicle of claim 1, wherein the hydrophobic core comprises the agent; and
b. administering the nanovesicle to the subject.

35. The method of claim 28, wherein the target area is a tumor.

36. (canceled)
